(19) United States
(12) Patent Application Publication
(10) Pub. No.: US 2007/0105835 A1
(43) Pub. Date: May 10, 2007

(54) Compositions and methods for modulating poly(ADP-ribose) polymerase activity

(76) Inventor: Aleksey G. Kazantsev, Brookline, MA (US)

Correspondence Address: FISH & RICHARDSON PC, P.O. BOX 1022, MINNEAPOLIS, MN, 55440-1022, US

(21) Appl. No.: 11/593,902

(22) Filed: Nov. 7, 2006

Related U.S. Application Data
(60) Provisional application No. 60/790,970, filed on Apr. 11, 2006.
(60) Provisional application No. 60/734,154, filed on Nov. 7, 2005.

(51) Int. Cl. A61K 31/553; A61K 31/551; A61K 31/365

(52) U.S. Cl. 514/211.04; 514/220; 514/450

(57) ABSTRACT

The present invention is based, in part, on assays we conducted that revealed compounds that modulate (e.g., inhibit) PARP-1 and are therefore useful in treating or preventing diseases characterized by abnormal PARP-1 activity (e.g., undesirable PARP-1 activity).

### RELATED APPLICATIONS

This application claims the benefit of U.S. application Ser. No. 60/734,154, filed Nov. 7, 2005, and U.S. application Ser. No. 60/790,970, filed Apr. 11, 2006. For the purpose of any U.S. patent that may issue from the present application, these two prior applications are hereby incorporated by reference herein in their entireties.

### TECHNICAL FIELD

This invention relates to compositions and methods for modulating poly(ADP-ribose) polymerase activity. We describe exemplary compounds, which may be contained in pharmaceutical compositions, the screening methods by which they were discovered, and their use as therapeutic or prophylactic agents.

### BACKGROUND

Poly(ADP-ribose) polymerase (PARP) is an enzyme that catalyzes the covalent attachment of ADP-ribose units from NAD(+) to various nuclear proteins. The most abundant and well-characterized member of the PARP family, PARP-1, includes an N-terminal zinc-dependent DNA-binding domain, a central automodification domain and a C-terminal NAD⁺-binding domain.

PARP-1 is involved in the regulation of cellular functions including differentiation, proliferation and tumor transformation, as well as the regulation of molecular events involved in the recovery of a cell from DNA damage. PARP-1 is also involved in the storage of cellular energy pools and transcriptional regulation of pro-inflammatory genes.

### SUMMARY

The present invention is based, in part, on our discovery of compounds that can be used to treat or prevent diseases that are associated with poly(ADP-ribose)polymerase-1 (PARP-1) activity within a cell. The compounds can, for example, be used in the treatment or prevention of disorders in which overexpression or unwanted expression of PARP-1 is associated. For example, the compounds can be used in the treatment or prevention of disorders that sensitize cells to oxidative stress, such as neurodegenerative diseases (e.g., Huntington's Disease (HD), Amyotrophic Lateral Sclerosis (ALS), and Alzheimer's disease), metabolic diseases (e.g., hereditary hemochromatosis), and cardiovascular diseases (e.g., atherosclerosis). Overexpression of PARP-1 has also been associated with various cancers, including human Ewing's sarcoma and high-grade lymphoma. PARP-1 is involved in promoting transcription of pro-inflammatory genes. Therefore, we expect inhibitors of PARP-1 can be used to downregulate multiple simultaneous pathways of inflammation and tissue injury such as those that are active in circulatory shock, colitis, and diabetic complications. Thus, inhibitors of PARP-1 can be used to treat inflammation (by, for example, delaying its onset or reducing its severity) in a variety of contexts, and the present compositions and methods are so intended. PARP-1 also has the ability to deplete cellular pools of ATP which can lead to parenchymal cell necrosis. Inhibitors of PARP-1 are therefore useful for reducing parenchymal cell necrosis, such as occurs in stroke and myocardial infarction. Given the relationship between breaks in DNA and the activation of PARP-1, it follows that PARP-1 inhibitors should enhance the cytotoxicity of certain DNA-damaging anti-cancer drugs, including temozolomide and bleomycin. Accordingly, PARP-1 inhibitors, including those described herein, can be administered with DNA-damaging anti-cancer drugs. Administration “with” encompasses administration of physically combined PARP-1 inhibitors and DNA-damaging anti-cancer drugs and administration of PARP-1 inhibitors and DNA-damaging anti-cancer drugs that are administered at, or at about, the same time by the same or different routes.

Certain compounds, which are described further below, were identified in our screening assays based on their ability to rescue HD cells from a pathological loss of ATP and subsequent cell death. While these compounds may affect transcription levels, cellular energy stores, and/or DNA repair mechanisms, whether by modulating PARP-1 activity through an upstream or downstream event, the invention is not limited to compounds that exert their effect on the disease process by any of these particular mechanisms. While we tend to use the term “compound(s),” we may also use terms like “agent(s)” to refer to the molecules (e.g., PARP-1 modulators, including PARP-1 inhibitors) described herein.

We have placed each of the compounds we identified into one of two categories. The compounds in the first category are represented by Formula I, and the compounds in the second category are represented by Formula II. The invention encompasses these compounds in, for example, a substantially pure form, as well as various compositions containing one or more of them (e.g., pharmaceutical formulations and concentrated stock solutions), salts, solvates, hydrates, or prodrugs thereof, and methods of using them.

Formula I is:

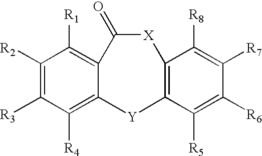

In Formula I, each of X and Y, independently, is O or NR₉; each of R₁, R₂, R₃, R₄, R₅, R₆, R₇, and R₈, independently, is R₁₀, halo, NR₁₁R₁₂, OR₁₀, C(O)R₁₀, C(O)OR₁₀, C(O)NR₁₁R₁₂, CN, or NO₂; R₉, independently, is H, alkyl, cycloalkyl, heterocycloalkyl, aryl, heteroaryl, or C(O)R₁₀; R₁₀, independently, is H, alkyl, cycloalkyl, heterocycloalkyl, aryl, or heteroaryl; each of R₁₁ and R₁₂ is, independently, H, alkyl, cycloalkyl, heterocycloalkyl, aryl, heteroaryl; or R₁₁ and R₁₂ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl. Specific compounds that conform to Formula I are shown in Table 1.

Formula II is:

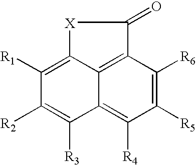

In Formula II, X is O or NR₇; each of R₁, R₂, R₃, R₄, R₅, and R₆, independently, is R₈, halo, NR₉R₁₀, OR₈, C(O)R₈, C(O)OR₈, C(O)NR₉R₁₀, CN, or NO₂; R₇ is H, alkyl, cycloalkyl, heterocycloalkyl, aryl, heteroaryl, or C(O)R₁₀; R₈, independently, is H, alkyl, cycloalkyl, heterocycloalkyl, aryl, or heteroaryl; each of R₉ and R₁₀ is, independently, H, alkyl, cycloalkyl, heterocycloalkyl, aryl, heteroaryl; or R₉ and R₁₀ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl. Specific compounds that conform to Formula II are shown in Table 2.

The invention features pharmaceutically acceptable salts, solvates, or hydrates of a compound of any of Formulas I or II, and prodrugs, metabolites, structural analogs, and other pharmaceutically useful variants thereof. These other variants may be, for example, complexes containing the compound and a targeting moiety, as described further below, or a detectable marker (e.g., the compound may be joined to a fluorescent compound or may incorporate a radioactive isotope). When in the form of a prodrug, a compound may be modified in vivo (e.g., intracellularly) after being administered to a patient or to a cell in culture. The modified compound (i.e., the processed prodrug) may be identical to a compound described herein and will be biologically active or have enough activity to be clinically beneficial. The same is true of a metabolite; a given compound may be modified within a cell and yet retain sufficient biological activity to be clinically useful.

Packaged products (e.g., sterile containers (e.g., a vial or blister pack) containing one or more of the compounds described herein and packaged for storage, shipment, or sale) and kits, including at least one compound of the invention and instructions for use, are also within the scope of the invention.

In one aspect, the invention features substantially pure preparations of the compounds described herein or combinations thereof. A naturally occurring compound is substantially pure when it is separated to some degree from the compound(s) or other entities (e.g., proteins, fats, or minerals) it is associated with in nature. For example, a naturally occurring compound described herein is substantially pure when it has been separated from at least 50%, 60%, 70%, 75%, 80%, 85%, 90%, 95%, 96%, 97%, 98%, 99% or more of the compound(s) or other moieties it is associated with in nature. While the compounds of the invention may be naturally occurring and may be purified using conventional techniques, they may also be non-naturally occurring and may be synthesized (naturally occurring compounds can be synthesized as well). Compounds prepared by chemical synthesis are substantially pure, as are compounds that have been separated from a library of chemical compounds. A substantially pure compound may be one that is separated from all the other members of the compound library or it may be one that has been separated to a limited extent (e.g., it may remain associated with a limited number (e.g., 1, 2, 3, 4, or 5-10) of other members of the library). A compound library is not a pharmaceutical or therapeutic composition, and one of ordinary skill in the art would understand that the methods of the invention would not be carried out with compound libraries.

Regardless of their original source or the manner in which they are obtained, the compounds of the invention can be formulated in accordance with their use. For example, the compounds can be formulated within compositions for application to cells in tissue culture or for administration to a patient. For example, the compounds can be mixed with a sterile, pharmaceutically acceptable diluent (such as normal saline or phosphate-buffered saline (PBS)). As noted below, and as known in the art, the type of diluent can vary depending upon the intended route of administration. The resulting compositions can include additional agents, such as preservatives, and/or other active ingredients (e.g., other therapeutic agents, including those presently known and used to treat the conditions described herein). The compounds may also be applied to a surface of a device (e.g., a catheter, stent, surgical mesh, or prosthesis) or contained within a pump, patch, or other drug delivery device.

The compounds featured in the invention can be formulated for use in cell culture and/or in vivo administration and supplied as reagents for research or for use in diagnostic assays, as described herein. For example, the compounds can be used in connection with animal models of the diseases described above (e.g., administered to a cell in which PARP-1 is overexpressed), and one can use such cellular or animal models to help determine a dose response profile and cellular toxicity for any given PARP-1 inhibitor.

The full-length PARP-1 polypeptide is 1014 amino acids long (see GenBank Accession No. P09874, Sep. 13, 2005) and includes an N-terminal DNA binding domain, including two Zinc fingers, and a caspase cleavage site within a nuclear localization signal (NLS). PARP-1 also includes an auto-poly(ADP)ribosylation domain from about residues 372 to 524, and a C-terminal catalytic domain extending from about residues 655 to 1014. The catalytic region includes an alpha helical domain from about residues 662 to 762 and an NAD⁺ binding site within residues 788 to 1014. Compounds featured in the invention can inhibit PARP-1 activity by binding to any particular region of the polypeptide or otherwise interfering with the ability of the polypeptide to function as it otherwise would.

In addition to determining the effect of a compound on PARP-1 activity (and, in animal models or clinical trials, the effect of a compound on the signs and symptoms of a disease or other phenotype), the assays or screens can include a step in which one determines cellular toxicity. One can also generate a dose response profile of putative assay hits and record the results in a screening database. A screening database, produced as described, is within the scope of the present invention.

In some embodiments, the compositions of the present invention can be administered to a subject having HD or a cancer, such as human Ewing's sarcoma or high grade lymphoma or another cancer known to exhibit PARP-1 overexpression (Menegazzi et al., Mol. Carcinog. 25:256-61, 1999). In other embodiments, the compositions of the present invention can be administered to a subject who has had (e.g., a human patient who has been diagnosed as having or as having had) or is at risk of having a stroke, a traumatic brain injury, Parkinson's disease, meningitis, hypoglycemia, a myocardial infarction, a cardiopulmonary bypass, ischemic cardiomyopathy, aortic banding-induced heart failure, diabetic cardiomyopathy, doxorubicin-induced myocardial failure, ageing-associated heart failure, diabetic endothelial dysfunction or other diabetes-related complication (e.g., diabetic neuropathy), hypertension, a balloon angioplasty, an endothelial injury (e.g., by homocysteine), interstitial pulmonary fibrosis, adult respiratory distress syndrome (ARDS), hyperoxic lung injury, ovalbumin-induced asthma, uveitis, diabetic retinopathy, optic nerve transaction, diabetes, colitis, mesenteric ischemia reperfusion, arthritis, reperfusion injury, organ transplantation, acoustic trauma to the ear, acetaminophen toxicity to the liver, sulphur mustard-induced vesication of the skin, cavernous nerve injury, an infection with HIV or a disease related thereto (e.g., AIDS, ARC, or Kaposi's sarcoma), or an ischemia reperfusion (I/R) injury (e.g., cochlear I/R, retinal I/R, or thoracoabdominal I/R). The inhibitors featured in the invention can also be used for the treatment or prevention of hemorrhagic, endotoxic, and septic shock.

Therapeutic methods featured in the invention can include the step of identifying a subject (e.g., a human patient of any age) in need of treatment. The subject can be identified by, for example, a health care professional (e.g., a physician) on the basis of subjective or objective information (e.g., based on comments from the subject, a physical examination, and/or on measurable parameters (i.e., diagnostic tests)). Subjects who are treated with one or more of the compounds described herein may have been diagnosed with any disease characterized by aberrant or undesirable PARP-1 expression, whether that expression occurs to a greater or lesser extent than is normal (in, e.g., a healthy patient) or desirable. Alternatively, the subject may be at risk for developing these disorders. For example, a subject may have a family history or a genetic mutation or element (e.g., an expanded trinucleotide repeat that may be indicative of the development of Huntington's disease) that contributes to the development of disease. Human subjects, in consult with their physicians and/or other health care professionals, can decide whether their risk is great enough to undergo preventative care (as is the case for any prophylactic treatment or procedure). While the subjects of the preventative and/or therapeutic regimes described herein may be human, the compounds and compositions of the invention can also be administered to non-human subjects (e.g., domesticated animals (such as a dog or cat), livestock (e.g., a cow, pig, sheep, goat, or horse), or animals kept in captivity (e.g., any of the large cats, non-human primates, zebra, giraffes, elephants, and the like kept in zoos, parks, or preserves)).

The prophylactic and therapeutic methods can be carried out by administering to the subject a pharmaceutical composition containing a therapeutically effective amount of one or more of the compounds described herein. While a single compound may be effective, the invention is not so limited. A subject can be treated with multiple compounds, administered simultaneously or sequentially (i.e., before or after a compound of the present invention). For example, a subject can be treated with one or more of the compounds described herein and, optionally, a chemotherapeutic agent, an analgesic, a bronchodilator, levodopa or a similar medication, haloperidol, or risperidone. In other embodiments, the “second” agent can be a vitamin, mineral, nucleic acid (e.g., an antisense oligonucleotide or siRNA), a therapeutic protein (e.g., a peptide), including therapeutic antibodies or antigen-binding portions thereof, or an anti-inflammatory agent. Compositions containing a compound featured in the invention and a second agent, as described herein, are also within the scope of the present invention.

The combination therapy will, of course, depend on the disorder being treated. Where a compound of the invention is administered to treat a patient with a cancer, it may be combined with a known chemotherapeutic agent or other form of treatment (e.g., a radiation-based therapy) used to treat that type of cancer. In another example, where a compound of the invention is administered to treat a patient with Huntington's disease (HD), it may be combined with a medication that acts as a dopamine blocker, such as haloperidol or phenothiazine. These and other combinations of active ingredients as well as combination therapies carried out by administering same are within the scope of the invention.

Compounds that interact with PARP-1 can also be used to diagnose diseases characterized by PARP-1 overexpression or hyperactivity. These methods can be carried out by providing a biological sample from a patient suspected of having a disease associated with PARP-1 overexpression or hyperactivity; exposing the sample to a compound of the invention; and determining whether the compound inhibits PARP-1 activity within the sample and, subsequently (or as a consequence), a sign or symptom of the disease is alleviated. For example, a sample will be exposed to the compound for a time and under conditions (e.g., physiological conditions of temperature and pH) sufficient to permit the compound to interact with PARP-1. The cell or animal that contains or constitutes the sample can then be analyzed for the presence or absence of a PARP-1-related disease or a diminution in a sign or symptom thereof. The diagnostic methods can be carried out before, after, or in conjunction with other diagnostic tests, and their results can inform the subject's treatment regime. For example, where a compound is found to inhibit PARP-1 activity in a sample obtained from a patient suspected of having a disease characterized by PARP-1 overexpression or hyperactivity, that compound may then be used to treat the patient.

Compounds that can modulate PARP-1 activity can be identified by the screening methods featured in the invention. As noted above, these compounds may modulate PARP-1 activity in different ways. The screening methods featured in the invention are not limited to those that identify compounds that work by any particular mechanism, nor are the compounds so limited. In some embodiments, the compounds may bind to PARP-1 polypeptides. In other embodiments, the compounds may act as transcriptional repressors or enhancers (in this scenario, a compound stimulates or inhibits transcription of the PARP-1 gene, or a gene encoding a polypeptide that interacts with the PARP-1 gene, or a PARP-1 RNA or a PARP-1 polypeptide). The compounds featured in the invention may also (or may alternatively) affect protein or RNA stability, thereby affecting polypeptide accumulation within a cell. The compounds may also modulate the post-translational processing of a protein. For example, a compound may interact with a kinase, phosphatase, methyl transferase, ubiquitinase, protease (e.g., an aspartyl protease such as cathepsin D or BACE-1 or BACE-2), or other modifying enzyme. Interruption of post-translational processing events may alter PARP-1 activity or the ability of PARP-1 to interact with other proteins or nucleic acids. Representative compounds are shown in Tables 1 and 2 below.

Other features and advantages of the invention will be apparent from the accompanying drawings and description, and from the claims.

### BRIEF DESCRIPTION OF THE DRAWINGS

FIGS. 1A-1D. FIG. 1A is a bar graph showing ATP levels in normal (white) and mutant HD (black) lymphoblasts, treated with the oxidant hydrogen peroxide. FIG. 1B is a graph showing relative ATP loss in normal (dashed line) and mutant HD (solid line) lymphoblasts, treated with the oxidant hydrogen peroxide. Basal ATP levels in untreated cells were 100%. A 50% loss of ATP was found to occur at oxidant concentrations of 120 μM and 60 μM for normal and mutant HD lymphoblasts respectively (dotted lines). FIG. 1C is a bar graph showing ATP levels in normal (white) and mutant HD (black) double knock-in striatal cells treated with hydrogen peroxide. FIG. 1D is a graph showing relative ATP loss in wildtype (dashed line) and mutant HD (solid line) striatal cells, treated with hydrogen peroxide. Basal ATP levels in untreated cells were 100%. A 50% loss of ATP was found to occur at oxidant concentrations of 550 μM and 420 μM for wildtype and mutant cells respectively (dotted lines).

FIGS. 2A-2F. FIG. 2A is a bar graph showing ATP levels in PC12 cells treated with the oxidant (Oxd.), hydrogen peroxide, in the absence (white) and in the presence (gray) of the PARP-1 inhibitor (ANI). FIG. 2B is a graph showing relative ATP levels in PC12 cells treated with hydrogen peroxide in the absence (solid line) and in the presence (dashed line) of 5 μM PARP-1 inhibitor ANI. FIG. 2C is a bar graph showing ATP levels in PC12 cells either untreated (U) or treated (T) with 200 μM oxidant and supplemented with the compounds indicated at the concentrations indicated. FIG. 2D is a panel showing the structures of the known PARP-1 inhibitors 3-AB, ANI, and AG 14361, the novel inhibitors CG1 and K245, and compound CG3. The IC₅₀ of each inhibitor, determined by in vitro assay, is also shown. The control compound CG3, failed to inhibit PARP-1 activity in vitro or in vivo. FIG. 2E is a bar graph showing the effect of different compounds on recombinant PARP-1 activity in vitro. PARP-1 enzymatic activities were determined in the absence (white) or presence (gray) of the testing compounds. Known PARP-1 inhibitor 3-AB and compound CG3 were used as positive and negative controls, respectively. FIG. 2F is a graph showing the calculated IC₅₀ of inhibitor K245. Compound concentrations were plotted against PARP-1 activity to establish an inhibition curve (solid line), and the IC₅₀ was determined to be 2 μM (dotted lines).

FIGS. 3A-3F. FIG. 3A is a bar graph showing the effect of the PARP-1 inhibitor ANI on ATP levels in one normal (white) and two mutant HD (black) lymphoid cell lines treated with the oxidant (Oxd.) hydrogen peroxide. Lymphoblasts treated (T) with 200 μM oxidant were supplemented with mock inhibitor (DMSO) or with 5 μM PARP-1 inhibitor ANI. FIG. 3B is a bar graph showing the effect of the novel PARP-1 inhibitor K245 on ATP levels in oxidant (Oxd.) treated normal (white) and mutant HD (black) lymphoid cell lines. Hydrogen peroxide was the oxidant. Lymphoblasts treated (T) with 200 μM oxidant were supplemented with mock inhibitor (DMSO) or with 1, 5 or 10 μM PARP-1 inhibitor K245. FIG. 3C is a bar graph showing the effects of various compounds on relative ATP levels in wildtype (white) and mutant HD (black) striatal cells treated with 400 μM hydrogen peroxide. Basal ATP levels in cells untreated with oxidant were 100%. Oxidant-treated cells were supplemented with mock (DMSO) or PARP-1 inhibitors ANI and K245 at concentrations 0.1 μM, 1 μM, and 5 μM; and 1 μM, 5 μM, and 10 μM, respectively. The inactive control compound CG3 was tested at concentrations of 1 μM, 5 μM, and 10 μM. FIG. 3D is a bar graph showing the effects of PARP-1 inhibitors ANI and K245 on basal ATP levels in normal (white) and mutant HD (black) lymphoblasts. Cells were supplemented with 5 μM ANI and 10 μM K245. Basal ATP levels in cells supplemented with mock (DMSO) were 100%. Cells were not treated with oxidant. FIG. 3E is a bar graph showing the effects of PARP-1 inhibitors on ATP levels in untreated striatal cells and in striatal cells treated with the mitochondrial blocker 3-NP. Wildtype cells are indicated by white bars and mutant HD cells are indicated by black bars. Untreated wildtype and mutant HD cells were supplemented with 5 μM ANI or 10 μM K245. Basal ATP levels in cells supplemented with mock (DMSO) were 100%. Cells treated with 5 μM 3-NP were supplemented with mock (DMSO), 1 or 5 μM ANI, or 1 μM, 5 μM, or 10 μM K245. FIG. 3F is a bar graph showing PARP-1 activity levels in untreated wildtype (7Q) and mutant HD (111Q) striatal cells, and in normal (30Q) and mutant HD (63Q) lymphoblasts.

FIG. 4 shows the structure of the K245 general scaffold and representative K245 structural analogs.

FIG. 5 shows the structure of representative K245 structural analogs.

FIG. 6 is a graph illustrating the effect of three different K245 analog inhibitors on PARP-1 activity.

### DETAILED DESCRIPTION

Small molecule-based therapeutics have provided the means to successfully treat many diseases, and the identification of pharmacological agents that can reverse, block, or delay disease-linked processes in model systems is critical to the development of effective treatments for the diseases described herein. Our assays employ in vitro model systems that recapitulate key features of disease pathology and that are adaptable to high throughput screening against a large collection of chemical compounds.

Using our assays and screens, we have identified compounds we believe are capable of inhibiting (either directly or indirectly) the activity of PARP-1. Inhibition of PARP-1 can protect cells from the pathological loss of energy stores (e.g., ATP stores), and subsequent cell death. These inhibitors are therefore appropriate for the treatment of diseases that are characterized by a decrease in cellular energy stores, e.g., HD, ALS, Alzheimer's Disease, and the other diseases referred to herein (we tend to use the term “disease” to refer to any disorder, unwanted condition, syndrome, or event).

Before describing exemplary compounds, we provide exemplary assays that can be used to test (or further test) those compounds as well as to identify other compounds or moieties, such as proteins (e.g., antibodies) and nucleic acids (e.g., oligonucleotides or molecules that mediate RNAi (e.g., siRNAs or shRNAs)) useful in the diagnosis, prevention, or treatment of a disease characterized by aberrant PARP-1 expression or by depletion of cellular ATP.

Assays: A variety of assays are available to identify, test and/or monitor the effect of a compound or other moiety on PARP-1 activity levels. In one assay, for example, an oxidant such as hydrogen peroxide is used to activate PARP-1 activity in vitro, such as in a cell culture. In vivo, oxidants and free radicals induce DNA breaks, and trigger PARP-1 activation. Activated PARP-1 cleaves NAD⁺ into nicotinomide and ADP-ribose, and uses the latter for extensive poly(ADP-ribosyl)ation of histones and other cellular proteins as a part of the DNA repair pathway. Ultimately, PARP-1 activity leads to the reduction of energy sources, including NAD⁺ and ATP, in cells. An assay for identifying a compound that inhibits PARP-1 activity can include treating a cell culture with an oxidant, such as hydrogen peroxide, and measuring ATP levels in the cell. A compound that reduces or eliminates the observed decrease in ATP levels upon treatment with the oxidant would be identified as an inhibitor.

In alternative (or additional) assays, cells can be treated with known activators of PARP-1 other than an oxidant. For example, cells can be exposed to a DNA alkylating agent (e.g., N-ethyl-N-nitrosourea (ENU), N-methyl-N-nitroso urea (MNU), or N-methyl-N′-nitro-N-nitrosoguanidine (MNNG)), UV irradiation, or any other means of inducing single or double-strand DNA breaks. A compound that reduces or eliminates the observed decrease in ATP levels upon treatment with the PARP-1 activator would be identified as an inhibitor and could be tested further in animal models or by clinical trials in human subjects (as is true of any of the compounds described herein).

ATP levels can be measured using known methods. For example, ATP levels can be measured using a luciferase substrate, such as luciferin. The luciferase enzyme uses ATP as a cofactor to produce one photon of light per luciferin molecule converted, and thus there is a linear relationship between the level of luminescence and the amount of ATP present in the test sample. Packaged luciferase assays are available through commercial suppliers and are also included in kits, such as the ATP Determination Kit from Molecular Probes (Invitrogen, Eugene, Oreg.). In an alternative approach, ATP levels can be measured by quantifying incorporation of labeled phosphate (e.g., ³²P) into ATP that is synthesized in a cell.

In another example, PARP-1 activity can be measured in vitro using cell extracts. PARP-1 activity can be assayed by measuring the level of poly-ADP ribose (PAR) incorporated onto histones. PAR levels can be measured, for example, by using a PAR-specific antibody.

In another example, PARP-1 activity can be assayed by incubating protein extracts containing PARP-1 (e.g., nuclear or mitochondrial extracts) with histones and labeled NAD+ (e.g., [³²P]NAD+ or [³H]NAD+) with and without exogenous DNA having strand breaks. PARP-1 activity is then measured by incorporation of the label into TCA-precipitatable proteins (see, e.g., Du et al., Jour. Biol. Chem. 278:18426-18433, 2003; and the PARP Activity Assay Kit from Trevigen (Gaithersburg, Md.)).

PARP-1 activity levels can also be measured by a similar method in vivo. In one exemplary assay, cells in culture are treated with an oxidant, such as hydrogen peroxide. After an incubation period, the medium is replaced with a PARP-1 assay buffer containing labeled NAD+ (e.g., biotinylated NAD+). After another short incubation period (e.g., 30 minutes at 37° C.), the buffer is removed and the cells fixed, such as with 95% ethanol. Cells are then blocked, such as with 1% bovine serum albumin (BSA), and the cells are contacted with a probe to detect the labeled NAD+. For example, horseradish peroxidase (HRP)-labeled streptavidin can be added to the cells, and the reaction developed with a peroxidase substrate, such as TACS-Sapphire™ (Trevigen, Gaithersburg, Md.).

By yet another method, a compound that causes a decrease in PARP-1 protein levels in a cell, indicating an inhibition of PARP-1 transcription or translation, can be identified as a PARP-1 inhibitor.

A compound that acts as an inhibitor in one assay will not necessarily exhibit an effect on PARP-1 activity in other assays. For example, a compound that inhibits a decrease in ATP levels following treatment of a cell with an oxidant may not exhibit a decrease in a PARP-1 activity assay or a decrease in PARP-1 protein levels. Similarly, a compound that exhibits an inhibitory effect in a PARP-1 activity assay or that causes a decrease in PARP-1 protein levels will not necessarily inhibit the oxidant-induced decrease in cellular ATP levels. PARP-1 inhibitors that prevent or weaken the oxidant-induced decrease in cellular ATP levels are preferred as potential therapeutic agents for the treatment of a disease marked by energy-deficient cellular stores, such as HD, ALS, Alzheimer's disease and the like.

Other compounds may inhibit different functions of PARP-1. For example, a compound may inhibit the role of PARP-1 as a transcriptional activator of pro-inflammatory genes. These compounds are potential therapeutic agents for the treatment of conditions or disorders that are a consequence of or are exacerbated by the activation of multiple simultaneous pathways of inflammation or tissue injury. For example, these compounds can be useful for the treatment of circulatory shock, colitis, and diabetic complications. Assays for identifying PARP-1 compounds that inhibit the ability of PARP-1 to activate transcription of pro-inflammatory genes include gene expression assays well-known in the art of molecular and cell biology. For example, fusing a PARP-1 responsive promoter or enhancer to a reporter gene and assaying for a decrease in reporter gene expression can be useful for the identification of such compounds. Genes known to be activated by PARP-1 include IL-1beta, tumor necrosis factor alpha, and inducible nitric oxide synthase (Ha, Proc Natl Acad Sci USA 101:5087-92, 2004, Epub. Mar. 23, 2004). Exemplary marker genes include green fluorescent protein (GFP), α-glucoronidase (GUS), luciferase, chloramphenicol transacetylase (CAT), horseradish peroxidase (HRP), alkaline phosphatase, acetylcholinesterase or β-galactosidase gene. Suitable expression control sequences can be selected by one of ordinary skill in the art. Standard methods can be used by the skilled person to construct expression vectors. See, generally, Sambrook et al., 1989, Cloning—A Laboratory Manual (2^{nd} Ed), Cold Spring Harbor Laboratory Press, Cold Spring Harbor, N.Y. Useful vectors include plasmid vectors and viral vectors. Viral vectors can be, for example, those derived from retroviruses, adenoviruses, adeno-associated virus, SV40 virus, pox viruses, or herpes viruses. Once introduced into a host cell (e.g., a bacterial cell, a yeast cell, an insect cell, an avian cell, or a mammalian cell), the vector can remain episomal, or be incorporated into the genome of the host cell. Useful vectors include vectors that can be purchased commercially, e.g., pcDNA 3.1-based vectors can be purchased from Invitrogen (Carlsbad, Calif.).

A compound can be virtually any substance (e.g., the compound can be a biological molecule, such as a polypeptide expressed in the cell, a chemical compound, or a synthetic nucleic acid (e.g., siRNA or antisense RNA) or polypeptide). Libraries that encode or contain candidate compounds are available to those of ordinary skill in the art through charitable sources (e.g., ChemBridge Corporation (San Diego, Calif.) (which provides useful information about chemical libraries on the worldwide web)) and commercial suppliers (e.g., TimTec, Newark, Del.).

The cells that can be used in the methods and assays described herein can be of any cell type that expresses PARP-1. For example, the cells can be mammalian cells (e.g., cells of a mouse or rat or other rodent, a non-human primate, or a human), fungal cells (e.g., yeast cells), insect cells (e.g., Drosophila cells), or worm cells (e.g., C. elegans cells). The cells can be wild type or mutant. For example, exemplary mutant cell lines include cells carrying an expanded polyglutamine (polyQ) repeat (e.g., a CAG or CAA trinucleotide repeat) in the huntingtin gene, and can be used to identify compounds that may be useful in the treatment of HD. The huntingtin gene in a mutant HD cell line can include for example, more than 30 CAG (or CAA, or a combination of CAA or CAG) trinucleotide repeats (e.g., 35, 40, 50, 60, 70, 80, 90, 100 or more trinucleotide repeats). The cells can be from any tissue type, including, neural cells (e.g., PC12 cells, a striatal cell line, or primary neurons) or cells from a tissue of the immune system (e.g., a lymphoblast cell line). In one assay, a candidate compound that may modulate (e.g., inhibit or promote) PARP-1 activity, is added to a cell culture that has been exposed to an oxidant, such as hydrogen peroxide. A compound that inhibits PARP-1 activity, and consequently prevents or inhibits depletion of ATP, will prevent cell death or will slow the death of the cells. In an alternative assay, or as an additional test of PARP-1 activity, cellular ATP levels can be measured. Cells treated with the oxidant typically experience a decrease in ATP levels as a consequence of PARP-1 activation. In the presence of an inhibitor compound, ATP levels will not decrease to the same extent, or will not decrease at all, compared to cells that are not administered the PARP-1 inhibitor compound.

Cultured cells can also be used to carry out toxicity studies of the compounds described herein and others (e.g., others identified by the assays featured in the invention). Compounds that undesirably block PARP-1 activity (e.g., prevent DNA double strand break repair to a certain extent) are likely to affect cell viability.

Alpha-synuclein toxicity assays can also be used to identify compounds that positively impact cell survival and/or function. We have observed small but reproducible protective effects of PARP-1 inhibitors, including K245, in a Parkinson's Disease α-synuclein toxicity assay. As noted, Parkinson's Disease is among the indications for the present compositions.

Compounds: The compounds that may be used as described herein are those conforming to Formula I or II, or prodrugs or biologically active variants of the compounds. When in the form of a prodrug, a compound may be modified in vivo (e.g., intracellularly) after being administered to a patient or to a cell in culture. The modified compound (i.e., the processed prodrug) may be identical to a compound described herein and will be biologically active or have enough activity to be clinically beneficial. The biologically active variants may be, for example, a complex containing the compound and a targeting moiety or a detectable marker (e.g., the compound may be joined to a fluorescent compound or may incorporate a radioactive isotope).

The above-described compounds can be used in, for example, a substantially pure form, as well as various compositions containing one or more of them (e.g., pharmaceutical formulations). The efficacy of these compounds can be assessed by assays such as those described above. Based on the results, appropriate dosage ranges and administration routes of these compounds can also be determined.

Examples of compounds of Formula I are shown in Table 1 below.

TABLE 1^{a}
Exemplary compounds of Formula 1.

| | |
|---|---|
| 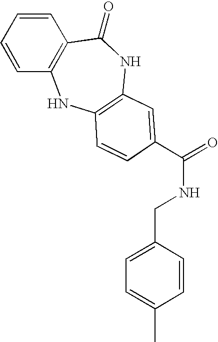 | 0036 |
| 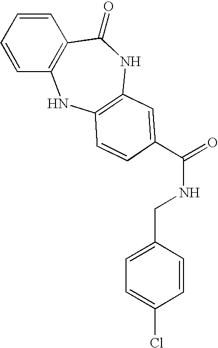 | 0073 |
| 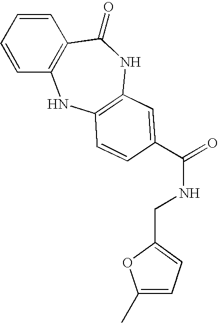 | 0076 |
| 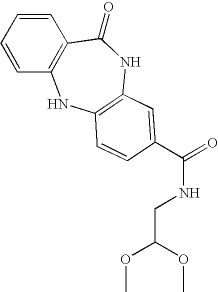 | 0095 |
| 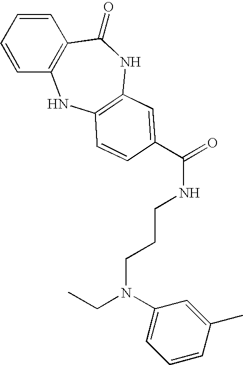 | 0099 |
| 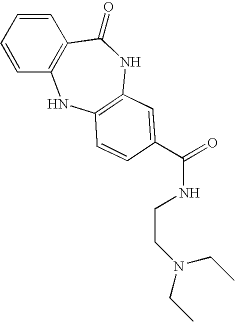 | 0014/K245 |
| 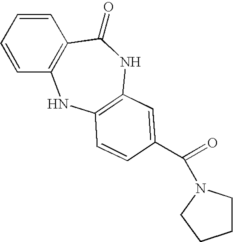 | 0080 |
| 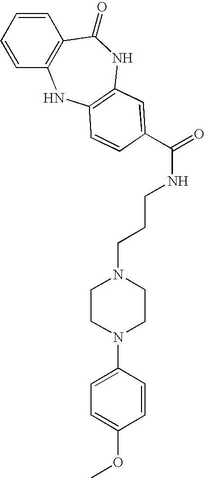 | 0039 |
| 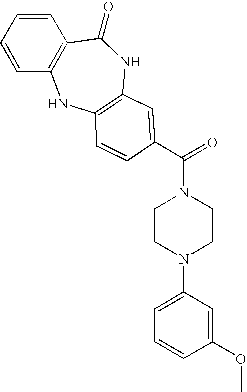 | 0084 |
| 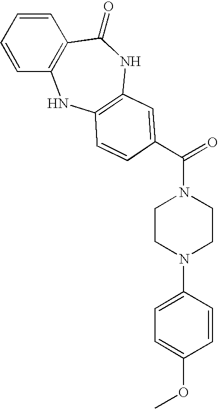 | 0039A |
| 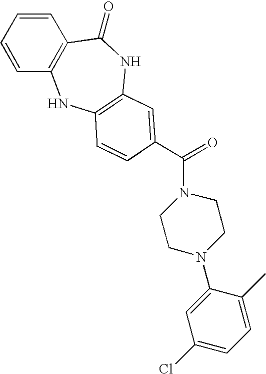 | 0071 |
| 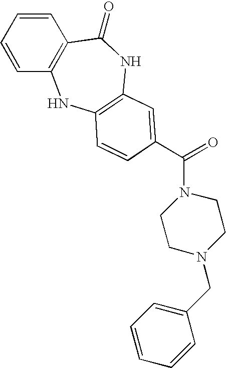 | 0042 |
| 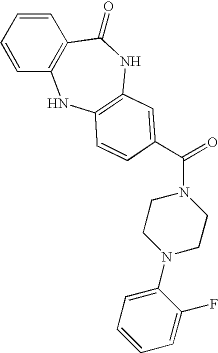 | 0089 |
| 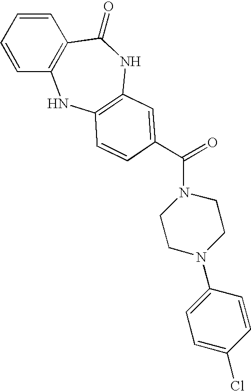 | 0088 |

^{a}Compounds are shown with their numerical identifiers.

Examples of compounds of Formula II are shown in Table 2 below.

TABLE 2
Exemplary compounds of Formula II

| | |
|---|---|
| 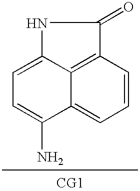 | 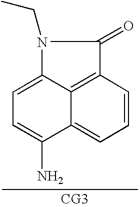 |

The compounds of Formulas I and II can have an IC₅₀ value as determined by in vitro or in vivo assay, and the value can range from about 0.10 to 6.0 μM or higher. For example, the IC₅₀ can be about 0.15 μM, about 0.5 μM, about 2.0 μM, about 3.0 μM, about 4.0 μM, or about 5.0 μM.

Compounds of Formulas I and II, including the compounds in Table 1 and Table 2 can be developed for the treatment of a subject who has, who has been diagnosed as having, or who is at risk of developing, a disorder characterized by unwanted PARP-1 expression. In certain embodiments, the compounds can be administered to a subject having HD or a cancer, such as human Ewing's sarcoma, or high grade lymphoma, which are cancers known to exhibit PARP-1 overexpression (Menegazzi et al., Mol. Carcinog. 25:256-61, 1999). The role of PARP-1 in the cellular processes described above (including its role in oxidative stress, pro-inflammatory gene expression, and its effect on cellular stores of ATP) suggests that the inhibitors featured in the invention can be useful for treating injuries and disorders of the brain (including stroke, traumatic brain injury, Parkinson's disease, meningitis, and hypoglycemia), injuries and disorders of the heart (including myocardial infarction, cardiopulmonary bypass, ischemic cardiomyopathy, aortic banding-induced heart failure, diabetic cardiomyopathy, doxorubicin-induced myocardial failure, and aging-associated heart failure), injuries and disorders of the vasculature (including diabetic endothelial dysfunction, hypertension, aging, balloon angioplasty, and endothelial injury by homocysteine), injuries and disorders of the lung (including interstitial pulmonary fibrosis, adult respiratory distress syndrome (ARDS), hyperoxic lung injury, and ovalbumin-induced asthma), injuries and disorders of the eye (including uveitis, diabetic retinopathy, and optic nerve transaction), diabetes, colitis, mesenteric ischemia reperfusion, arthritis, reperfusion injury, organ transplantation, acoustic trauma to the ear, acetaminophen toxicity to the liver, sulphur mustard-induced vesication of the skin, diabetic neuropathy, cavernous nerve injury, HIV, ischemia reperfusion (I/R), e.g., cochlear I/R, retinal I/R, or thoracoabdominal I/R, hemorrhagic, endotoxic, and septic shock. Potential therapeutic targets of PARP inhibitors are reviewed in Jagtap and Szabo (Nature Reviews 4:421-440, 2005).

As noted above, while we tend to use the term “compound(s),” we may also use terms like “agent(s)” to refer to the molecules described herein. The following definitions apply to the terms used in connection with any of the formulas described herein. The term “halo” or “halogen” refers to any radical of fluorine, chlorine, bromine or iodine. The terms “cyclylalkyl” and “cycloalkyl” refer to saturated monocyclic, bicyclic, tricyclic, or other polycyclic hydrocarbon groups. Any atom can be substituted by, for example, one or more substituents. Cycloalkyl groups can contain fused rings, which share a common carbon atom. Cycloalkyl moieties can include, for example, cyclopropyl, cyclohexyl, methylcyclohexyl (the point of attachment to another moiety can be either the methyl group or a cyclohexyl ring carbon), adamantyl, and norbornyl.

The term “alkenyl” refers to a straight or branched hydrocarbon chain containing 2-20 carbon atoms and having one or more double bonds. Any atom can be substituted by one or more substituents. Alkenyl groups can include, for example, allyl, propenyl, 2-butenyl, 3-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be substituted by one or more substituents. Alkynyl groups can include, for example, ethynyl, propargyl, and 3-hexynyl. One of the triple bond carbons can optionally be the point of attachment of the alkynyl substituent.

The term “alkoxy” refers to an —O-alkyl radical. The term “heterocyclyl” refers to a monocyclic, bicyclic, tricyclic or other polycyclic ring system having: 1-4 heteroatoms if monocyclic; 1-8 heteroatoms if bicyclic; or 1-10 heteroatoms if tricyclic. The heteroatoms can be O, N, or S (e.g., carbon atoms and 1-4, 1-8, or 1-10 heteroatoms of N, O, or S if monocyclic, bicyclic, or tricyclic, respectively). The heteroatom can optionally be the point of attachment of the heterocyclyl substituent. Any atom can be substituted, by, for example, one or more substituents. The heterocyclyl groups can contain fused rings, which share a common carbon atom. Heterocyclyl groups can include, for example, tetrahydrofuranyl, tetrahydropyranyl, piperidinyl, morpholino, pyrrolinyl, and pyrrolidinyl.

The term “heteroaryl” refers to an aromatic monocyclic, bicyclic, tricyclic, or other polycyclic hydrocarbon groups having: 1-4 heteroatoms if monocyclic; 1-8 heteroatoms if bicyclic; or 1-10 heteroatoms if tricyclic. The heteroatoms can be O, N, or S (e.g., carbon atoms and 1-4, 1-8, or 1-10 heteroatoms of N, O, or S if monocyclic, bicyclic, or tricyclic, respectively). Any atom can be substituted by, for example, one or more substituents. Heteroaryl groups can contain fused rings, which share a common carbon atom. Heteroaryl groups include pyridyl, thienyl, furanyl, imidazolyl, and pyrrolyl.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on, for example, an alkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituents on a group are independently any one single, or any subset of, the aforementioned substituents. In another aspect, a substituent may itself be substituted with any one of the above substituents.

Salts, solvates, hydrates and other variants: The invention also encompasses pharmaceutically acceptable salts or solvates of a compound of any of Formulas I and II, and prodrugs, metabolites, structural analogs, and other pharmaceutically useful variants thereof. These other variants may be, for example, a complex containing the compound and a targeting moiety, as described further below, a second therapeutic agent or a detectable marker (e.g., the compound may incorporate a radioactive isotope or be joined to a fluorescent compound). When in the form of a prodrug, a compound may be modified in vivo (e.g., intracellularly) after being administered to a patient or to a cell in culture. The modified compound (i.e., the processed prodrug) may be identical to a compound described herein and will be biologically active or have enough activity to be clinically beneficial. The same is true of a metabolite; a given compound may be modified within a cell and yet retain sufficient biological activity to be clinically useful.

A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion.

Examples of prodrugs include esters and other pharmaceutically acceptable derivatives, which, upon administration to a subject, are capable of providing active compounds. A “prodrug” may be any pharmaceutically acceptable salt, ester, salt of an ester, or other derivative of a compound of this invention (for example an imidate ester of an amide), which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selected biological properties (e.g., targeting to a particular tissue). Such modifications are known in the art and include those which increase biological penetration into a given biological compartment (e.g., blood, lymphatic system, central nervous system), increase oral availability, increase solubility to allow administration by injection, alter metabolism and alter rate of excretion.

The compounds of the invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) wherein bond rotation is restricted about that particular linkage (e.g., restriction resulting from the presence of a ring or double bond). Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

As noted, the compounds of the invention may be mixed with or joined to a detectable marker or tag, to another therapeutic agent, or to a moiety that facilitates passage across the blood-brain barrier (see below).

Packaged products: The compounds described herein can be packaged in suitable containers labeled, for example, for use as a therapy to treat a disease or disorder characterized by abnormal or undesired PARP-1 activity. The containers can include the compound (i.e., the diagnostic/prophylactic/therapeutic agent) and one or more of a suitable stabilizer, carrier molecule, flavoring, and/or the like, as appropriate for the intended use. Accordingly, packaged products (e.g., sterile containers containing one or more of the compounds described herein and packaged for storage, shipment, or sale at concentrated or ready-to-use concentrations) and kits, including at least one compound of the invention and instructions for use, are also within the scope of the invention. A product can include a container (e.g., a vial, jar, bottle, bag, or the like) containing one or more compounds of the invention and a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefore, and other uses. The compounds can be ready for administration (e.g., present in dose-appropriate units), and may include a pharmaceutically acceptable adjuvant, carrier or other diluent and/or an additional therapeutic agent. Alternatively, the compounds can be provided in a concentrated form with a diluent and instructions for dilution.

Stability: Combinations of substituents and variables envisioned by this invention are only those that result in the formation of stable compounds. The term “stable,” as used herein, refers to compounds that are stable enough to allow manufacture and that maintain their integrity for a sufficient period of time to be useful for the purposes detailed herein (e.g., therapeutic or prophylactic administration to a subject).

Purity: In one aspect, the invention features substantially pure preparations of the compounds described herein or combinations thereof. A naturally occurring compound is substantially pure when it is separated to some degree from the compound(s) or other entities (e.g., proteins, fats, or minerals) it is associated with in nature. For example, a naturally occurring compound described herein is substantially pure when it has been separated from 50%, 60%, 70%, 75%, 80%, 85%, 90%, 95%, 96%, 97%, 98%, 99% or more of the compound(s) or other moieties it is associated with in nature. These degrees of purity are not limiting, however, the compounds of the invention need be only as pure as necessary to cause a beneficial clinical result and to conform with good manufacturing practices. While the compounds of the invention may be naturally occurring and may be purified using conventional techniques, they may also be non-naturally occurring and may be synthesized (naturally occurring compounds can be synthesized as well; see below). Compounds prepared by chemical synthesis are substantially pure, as are compounds that have been separated from a library of chemical compounds. A substantially pure compound may be one that is separated from all the other members of the compound library or it may be one that has been separated to a limited extent (e.g., it may remain associated with a limited number (e.g., 1, 2, 3, 4, or 5-10) of other members of the library. As noted, while more than one of the agents described herein can be formulated within the same composition, and while the compositions can also include a second therapeutic agent (as described herein), the pharmaceutical compositions of the invention expressly exclude extremely heterogeneous mixtures, such as libraries (e.g., combinatorial or compound libraries, including those that contain synthetic and/or natural products, and custom analog libraries, which may contain compounds based on a common scaffold). Such libraries can include hundreds or thousands of distinct compounds or random pools thereof. Whether or not commercially available, such libraries are excluded from the meaning of a pharmaceutical composition.

Formulations: Regardless of their original source or the manner in which they are obtained, the compounds of the invention can be formulated in accordance with their use. For example, the compounds can be formulated within compositions for application to cells in tissue culture or for administration to a patient. For example, the compounds can be mixed with a sterile, pharmaceutically acceptable diluent (such as normal saline). As noted below, and as known in the art, the type of diluent can vary depending upon the intended route of administration. The resulting compositions can include additional agents, such as preservatives. The compounds may also be applied to a surface of a device (e.g., a catheter) or contained within a pump, patch, or other drug delivery device. The therapeutic agents of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formularly).

A pharmaceutical composition (e.g., a composition containing a therapeutic agent or the DNA molecule encoding it) is formulated to be compatible with its intended route of administration. Examples of routes of administration include oral, rectal, and parenteral, for example, intravenous, intradermal, and subcutaneous, transdermal (topical), and transmucosal administration. Variants of the compounds described herein, formulated to cross the blood-brain barrier, are described below.

Diagnostic, prophylactic and therapeutic use: The compounds identified by the methods described herein (which may also be referred to herein as “therapeutic agents”) may be used to treat a variety of disorders, including any disorder characterized by aberrant or unwanted PARP-1 activity. For example, the compounds described herein can be included as therapeutic agents in pharmaceutical compositions to treat HD.

Treating a subject can encompass administration of a therapeutic agent as a prophylactic measure to prevent the occurrence of disease or to lessen the severity or duration of the symptoms associated with the disease. Physicians and others of ordinary skill in the art routinely make determinations as to the success or failure of a treatment. Treatment can be deemed successful despite the fact that not every symptom of the disease is totally eradicated. Treatment can also be deemed successful despite side-effects.

It is usual in the course of developing a therapeutic agent that tests of that agent in vitro or in cell culture are followed by tests in animal models of human disease, and further, by clinical trials for safety and efficacy in humans. Accepted animal models for many diseases are now known to those of ordinary skill in the art. For example, therapeutic agents of the present invention can be screened in a Drosophila model of neurodegeneration as well as in more evolutionarily advanced animals.

Mammalian models for Huntington's disease are available. To generate similar animal models, a homolog of the huntingtin polypeptide is first cloned from the genome of the selected mammal using standard techniques. For example, the sequence can be amplified by PCR or obtained by screening an appropriate library under conditions of low stringency (as described, e.g., in Sambrook et al. supra.). Subsequently, trinucleotide repeats can be introduced into the gene by molecular cloning and mutagenesis techniques. For example, in a HD model, CAG repeats can be introduced in the HD gene. The site for insertion of the repeat sequence can be located by alignment of the cDNA from the desired mammal with the human cDNA for the huntingtin protein. The modified gene with artificially expanded repeats can be reintroduced into the mammal using standard methods for transgenesis.

Methods for generating transgenic mice are routine in the art (See, e.g., Hogan et al., Manipulating the Mouse Embryo, Cold Spring Harbor Laboratory Press, Cold Spring Harbor, N.Y. (1994)). As an example, a mouse bearing a transgene comprising the HD gene and expanded CAG repeats has symptoms similar to the human disease. Murine symptoms can include hyperactivity, circling, abnormal gait, tremors, learning deficits, hypoactivity, and hypokinesis. Neuropathological symptoms include general brain atrophy, progressive striatal atrophy, neuropil aggregates, inclusions in the striatum, reduced dendritic spines, and cell loss in the cortex and striatum.

Any of these behavioral or physiological deficits can be assessed in order to determine the efficacy of a given therapeutic compound featured in the invention. For example, the compound can be administered to a transgenic mouse model, generated as described above. The symptoms of a treated mouse can be compared to untreated mice at various times during and after treatment. In addition, treated and untreated mice can be sacrificed at various intervals after treatment, and the neuropathology of the brain can be analyzed. Thus, the efficacy of the treatment can be evaluated readily by comparing the behavioral symptoms, neuropathological symptoms, and clinical symptoms of treated and untreated mice.

In specific embodiments, the compositions of the present invention can be administered to a subject having any disease mediated by (or characterized by) abnormal or undesired PARP-1 activity.

Subjects who are treated with the compounds of the invention may have been diagnosed with any disease mediated by (or characterized by) abnormal or undesired PARP-1 activity, whether that activity occurs to a greater or lesser extent than is normal (in, e.g., a healthy patient) or desirable. Alternatively, the subject may be at risk for developing these disorders. For example, a subject may have a family history or a genetic mutation or element (e.g., an expanded trinucleotide repeat) that contributes to the development of disease. Human subjects, in consult with their physicians and/or other health care professionals, can decide whether their risk is great enough to undergo preventative care (as is the case for any prophylactic treatment or procedure). While the subjects of the preventative and/or therapeutic regimes described herein may be human, the compounds and compositions of the invention can also be administered to non-human subjects.

The prophylactic and therapeutic methods can be carried out by administering to the subject a pharmaceutical composition containing a therapeutically effective amount of one or more of the compounds described herein. While a single compound may be effective, the invention is not so limited. A subject can be treated with multiple compounds, administered simultaneously or sequentially. For example, a subject can be treated with one or more of the compounds described herein and, optionally, a chemotherapeutic agent, an analgesic, a bronchodilator, levodopa or a similar medication. The combination therapy will, of course, depend on the disorder being treated. Where a compound of the invention is administered to treat a patient with Parkinson's disease, it may be combined with a medication to increase dopamine levels in the brain. Where a compound of the invention is administered to treat a patient with a cancer, it may be combined with a known chemotherapeutic agent used to treat that type of cancer. Because PARP-1 is activated by DNA breaks, PARP-1 inhibitors can enhance the cytotoxicity of certain DNA-damaging chemotherapeutics, such as temozolomide and bleomycin.

Compounds that modulate PARP-1 activity can also be used to diagnose diseases characterized by such activity. These methods can be carried out by providing a biological sample from a patient suspected of having a disease associated with an abnormal or undesirable PARP-1 activity; exposing the sample to a compound of the invention; and determining whether the compound modulates the activity of PARP-1 within the sample. The compound can be one that is known to interact directly with a PARP-1 or one that modulates PARP-1 activity by acting along the biological pathway (e.g., upstream from the polypeptide). For example, a compound that is known to inhibit PARP-1 activity can be used to diagnose a patient suspected of having HD. The sample will be exposed to the compound for a time and under conditions (e.g., physiological conditions of temperature and pH) sufficient to permit the compound to affect PARP-1 activity within the sample. The diagnostic methods can be carried out before, after, or in conjunction with other diagnostic tests, and their results can inform the subject's treatment regime. For example, where a compound is found to modulate PARP-1 activity, or a PARP-1-induced phenotype (e.g., where the compound is found to inhibit the loss of ATP stores in a sample treated with an oxidant), that compound may then be used to treat the patient.

The blood-brain barrier is an obstacle for the delivery of drugs from circulation in the bloodstream to the brain. The endothelial cells of brain capillaries are connected by tight intercellular junctions, which inhibit the passive movement of compounds out of the blood plasma into the brain. These cells also have reduced pinocytic vesicles in order to restrict the indiscriminate transport of materials intracellularly. These features of the brain regulate the exchange of materials between plasma and the central nervous system. Both active and passive transport mechanisms operate to exclude certain molecules from traversing the barrier. For example, lipophilic compounds are more permeable to the barrier than hydrophilic compounds (Goldstein et al., Scientific American 255:74-83, 1996; Pardridge et al., Endocrin. Rev. 7:314-330, 1996).

However, the blood-brain barrier must also allow for the selective transport of desired materials into the brain in order to nourish the central nervous system and to remove waste products. The mechanisms by which this is accomplished can provide the means for supplying the therapeutic agents described herein.

The compositions of the invention can be delivered to the CNS following conjugation with other compounds as follows (and as described further in, for example, U.S. Pat. No. 5,994,392). In one instance, polar groups on a compound are masked to generate a derivative with enhanced lipophilic qualities. For example, norepinephrine and dopamine have been modified with diacetyl and triacetyl esters to mask hydroxyl groups. An implementation of this strategy has been previously used to create a pro-drug derivative of dopamine (see U.S. Pat. No. 5,994,392). The modified drugs are generally referred to as prodrugs, and the compounds of the invention encompass those described herein in which polar groups are masked. This method may have the additional advantage of providing an inactive species of the compound in the general circulation. After crossing the blood-brain barrier, enzymes present in the central nervous system are able to hydrolyze the linkages (e.g., ester linkages), thereby unmasking the compound and liberating the active drug. Thus, compounds of the invention can be chemically modified to create pro-drugs by, e.g., conjugation to a lipophilic moiety or carrier. A compound or a variant thereof having at least one free hydroxyl or amino group can be coupled to a desired carrier (e.g., a fatty acid, a steroid, or another lipophilic moiety).

More specifically, and for example, the hydroxyl groups can first be protected with acetonide. The protected agent is then reacted with the desired carrier in the presence of a water-extracting compound (e.g., dicyclohexyl carbodiiamide), in a solvent (e.g., dioxane, tetrahydrofurane), or N,N dimethylformamide at room temperature. The solvent is then removed, and the product is extracted using methods routinely used by those of ordinary skill in the art. Amine groups can be coupled to a carboxyl group in the desired carrier. An amide bond is formed with an acid chloride or low carbon ester derivative of the carrier. Bond formation is accompanied by HCl and alcohol liberation. Alcohol groups on the compound can be coupled to a desired carrier using ester bonds by forming an anhydride derivative, i.e., the acid chloride derivative, of the carrier. One of ordinary skill in the art of chemistry will recognize that phosphoramide, sulfate, sulfonate, phosphate, and urethane couplings are also useful for coupling a therapeutic agent (e.g., a compound described herein) to a desired carrier. A useful and adaptable method for lipidation of antibodies is described by Cruikshank et al. (J. Acquired Immune Deficiency Syndromes and Human Retrovirology 14:193, 1997).

Procedures for delivering therapeutic agents (or “compounds”) of the invention to the CNS can also be carried out using the transferrin receptor as described, for example, in U.S. Pat. No. 6,015,555. To implement this procedure, the agents are conjugated to a molecule that specifically binds to the transferrin receptor (e.g., an antibody or antigen-binding fragment thereof, or transferrin). Methods for obtaining antibodies against the transferrin receptor and for coupling the antibodies to a desired compound are also described in U.S. Pat. No. 6,015,555.

Monoclonal antibodies that specifically bind to the transferrin receptor include OX-26, T58/30, and B3/25 (Omary et al., Nature 286:888-891, 1980), T56/14 (Gatter et al., J. Clin. Path. 36:539-545, 1983), OKT-9 (Sutherland et al., Proc. Natl. Acad. Sci. USA 78:4515-4519, 1981), L5.1 (Rovera, Blood 59:671-678, 1982) and 5E-9 (Haynes et al., J. Immunol. 127:347-351, 1981). In one embodiment, the monoclonal antibody OX-26 is used. The antibody of choice can be a Fab fragment, a F(ab′)₂ fragment, a humanized antibody, a chimeric antibody, or a single chain antibody.

The antibody to the transferrin receptor is conjugated to a desired compound with either a cleavable or non-cleavable linker. The preferred type of linker can be determined without undue experimentation by making cleavable and non-cleavable conjugates and assaying their activity in, for example, an in vitro or cell culture assay described herein. The conjugates can be further tested in vivo (e.g., in a animal model of a disease of interest). Examples of chemical systems for generating non-cleavable linkers include the carbodiimmide, periodate, sulfhydryl-maleimide, and N-succinimidyl-3-(2-puridyldithio) propionate (SPDP) systems. Carbodiimide activates carboxylic acid groups, which then react with an amino group to generate a non-cleavable amide bond. This reaction may be especially useful for coupling two proteins. Periodate is used to activate an aldehyde on an oligosaccharide group such that it can react with an amino group to generate a stable conjugate. Alternatively, a hydrazide derivative of the desired compound can be reacted with the antibody oxidized with periodate. Sulfhydryl-maleimide and SDPD use sulfhydryl chemistry to generate non-cleavable bonds. SDPD is a heterobifunctional crosslinker that introduces thiol-reactive groups. In the sulfhydryl-maleimide system, an NHS ester (e.g., gamma-maleimidobutyric acid NHS ester) is used to generate maleimide derivative, for example, of a protein drug or antibody. The maleimide derivative can react with a free sulfhydryl group on the other molecule.

Cleavable linkers are also useful. Cleavable linkers include acid labile linkers such as cis-aconitic acid, cis-carboxylic alkadienes, cis-carboxylic alkatrienes, and polypeptide-maleic anhydrides (see U.S. Pat. No. 5,144,011).

In one embodiment, the compound is a compound having one of the structures shown in Tables 1 or 2. Such a compound can be covalently attached to an antibody specific for the transferrin receptor. In one embodiment, use of a single chain antibody is preferred in order to facilitate covalent fusion with the therapeutic agent.

The targeting antibody can be linked covalently to the therapeutic agent (or “compound”) of the invention. A protease recognition site can be included in the linker if cleavage of the antibody is required after delivery.

The efficacy of strategies to deliver a desired compound across the blood-brain barrier can, of course, be monitored. The desired compound, conjugated for delivery across the blood-brain barrier, is administered to a test mammal (e.g., a rat, a mouse, a non-human primate, a cow, a dog, a rabbit, a cat, or a sheep). One of ordinary skill in the art will, however, recognize that the permeability of the blood-brain barrier varies from species to species, with the human blood-brain barrier being the least permeable. The mode of administration can be the same as the desired mode of treatment (e.g., intravenous). For a comprehensive analysis, a set of test mammals is used. The test mammals are sacrificed at various times after the agent is administered and are then perfused through the heart with, e.g., Dulbecco's phosphate-buffered saline (DPBS) to clear the blood from all organs. The brain is removed, frozen in liquid nitrogen, and subsequently sectioned in a cryostat. The sections are placed on glass microscope slides. The presence of the desired agent is then detected in the section, for example with an antibody, or by having administered a radiolabeled or otherwise tagged compound (such labeled therapeutic compounds as described above). Detection is indicative of the compound having successfully traversed the blood-brain barrier. If a method of enhancing the compound's permeability to the blood-brain barrier is being assessed, then the amount of the agent detected in a brain section can be compared to the amount detected in a brain section from an animal treated with the same compound without the enhancing method.

The terms “blood-brain barrier permeant” or “blood-brain barrier permeable” are qualities of a compound for which the ratio of a compound's distribution at equilibrium in the cerebrospinal fluid (CSF) relative to its distribution in the plasma (CSF/plasma ratio) is greater than at least (or about) 0.01, 0.02, 0.05, or 0.1. While lower ratios are generally preferred, any ratio that allows a compound to be used clinically is acceptable.

To facilitate targeting to a polypeptide of interest (e.g., to a PARP-1 or a protein or nucleic acid that interacts with PARP-1), the compound (e.g., a compound conforming to any of Formulas I or II) can include a moiety that specifically binds to the target protein. For example, a compound conforming to Formula I can be joined to an antibody or an antigen-binding portion thereof (e.g., a single chain antibody) that specifically binds the target protein (e.g., PARP-1). Targeting moieties are described further below.

A therapeutic vector can be administered to a subject, for example, by intravenous injection, by local administration (see U.S. Pat. No. 5,328,470) or by stereotactic injection (see e.g., Chen et al., Proc. Natl. Acad. Sci. USA 91:3054-3057, 1994). The compound can be further formulated, for example, to delay or prolong the release of the active agent by means of a slow release matrix.

Regardless of whether or not the compound is to cross the blood-brain barrier, it can be conjugated to a targeting agent that facilitates interaction with a target protein (e.g., PARP-1 or protein that interacts with PARP-1). As noted, the compound can be directly or indirectly joined to an antibody (e.g., a single chain antibody) or an antigen-binding fragment thereof that specifically binds the target protein.

An appropriate dosage of the therapeutic agents of the invention must be determined. An effective amount of a therapeutic compound is the amount or dose required to ameliorate a symptom of a disorder associated with unwanted PARP-1 activity, such as a disorder characterized by an enhanced cellular sensitivity to oxidative stress. Determining the amount required to treat a subject is routine to one of ordinary skill in the art (e.g., a physician, pharmacist, or researcher). First, the toxicity and therapeutic efficacy of an agent (i.e., a tri-domain molecule) is determined. Routine protocols are available for determining the LD₅₀ (the dose lethal to 50% of the population) and the ED₅₀ (the dose therapeutically effective in 50% of the population) in non-human animals. The therapeutic index is measured as the ratio of the LD₅₀/ED₅₀. Compounds, formulations, and methods of administration with high therapeutic indices are preferable as such treatments have little toxicity at dosages that provide high efficacy. Compounds with toxic or undesirable side effects can be used, if means are available to deliver the compound to the affected tissue, while minimizing damage to unaffected tissue.

In formulating a dosage range for use in humans, the effective dose of a therapeutic agent can be estimated from in vitro cell studies and in vivo studies with animal models. If an effective dose is determined for ameliorating a symptom in cell culture, a dose can be formulated in an animal in order to achieve a circulating plasma concentration of sodium butyrate that falls in this range. An exemplary dose produces a plasma concentration that exceeds the IC₅₀ (i.e., the concentration of the test compound which achieves a half-maximal inhibition of symptoms) as determined in cell culture assays. For example, an exemplary dose can produce a plasma concentration that exceeds an IC₅₀ of from about 0.10 to 6.0 μM (e.g., about 0.15 μM, about 0.5 μM, about 2.0 μM, about 3.0 μM, about 4.0 μM, about 5.0 μM). The circulating plasma concentration can be determined, for example, by administering a labeled therapeutic composition to the test animal, obtaining a blood sample, and quantitating the amount of labeled compound present at various times after administration.

An appropriate daily dose of a therapeutic agent can be between about 0.1 mg/kg of body weight to about 500 mg/kg, or between about 1 mg/kg to about 100 mg/kg. The dose can be adjusted in accordance with the blood-brain barrier permeability of the compound. For example, a therapeutic compound can be administered at a dosage of 50 mg/kg to 100 mg/kg in order to treat the brain. The dose for a patient can be optimized while the patient is under care of a physician, pharmacist, or researcher. For example, a relatively low dose of a tri-domain therapeutic can be administered initially. The patient can be monitored for symptoms of the disorder being treated (e.g., HD). The dose can be increased until an appropriate response is obtained. In addition, the specific dose level for any particular subject can vary depending on the age, body weight, general health, gender, and diet of the subject, the time of administration, the route of administration, the rate of excretion, and other drugs provided in combination.

As occurs in the course of all drug development, optimal treatment regimes will emerge through further modeling and clinical trials. It may be, for example, that a patient will receive a combination of compounds that act synergistically to inhibit PARP-1 activity by the same or different mechanisms of action. Combination therapies may also rely on administration of a compound that interferes with gene transcription (e.g., a small molecule or a nucleic acid that mediates RNAi) and a compound that facilitates degradation of any remaining unwanted polypeptide-containing complexes.

The efficacy of a dose of any therapeutic agent can be determined in a subject. For example, the subject can be monitored for clinical symptoms, for example, a symptom of a trinucleotide repeat disease, such as a symptom of HD. Behavioral symptoms of HD include irritability, apathy, lethargy, depression, hostile outbursts, loss of memory and/or judgment, loss of ability to concentrate, anxiety, slurred speech, difficulty swallowing and/or eating, and inability to recognize persons. Clinical symptoms of HD include loss of coordination, loss of balance, inability to walk, uncontrolled movements of the fingers, feet, face, and/or trunk, rapid twitching, tremors, chorea, rigidity, and akinesia (severe rigidity).

While the compounds featured in the invention include inhibitors of PARP-1, compounds of Formula I and Formula II also include compounds capable of enhancing PARP-1 function. PARP-1 deficiencies have been found to be associated with cancers, such as cancers of the colon, prostate and liver, and premature or rapid aging. Compounds identified as PARP-1 enhancers can be used to treat any disease or disorder characterized by decreased or inadequate PARP-1 activity. For example, such compounds can be used to treat cancer or to slow the degenerative effects of aging.

Methods of making: The compounds described herein can be synthesized using routine techniques known to one of ordinary skill in the art. For example, the compounds can be made by providing a starting compound or intermediate and reacting the compound or intermediate with one or more chemical reagents in one or more steps to produce a compound described herein (e.g., a compound of Formulas I or II). The compound can be separated from a reaction mixture and further purified by a method such as column chromatography, high-pressure liquid chromatography, or recrystallization. As can be appreciated by one of ordinary skill in the art, further methods of synthesizing the compounds of the formulae herein are available. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Techniques useful for the separation of isomers, for example, stereoisomers are within skill of the art and are described in Eliel, E. L.; Wilen, S. H.; Mander, L. N. Stereochemistry of Organic Compounds, Wiley Interscience, NY, 1994. For example compounds can be resolved via formation of diasteromeric salts, for example, with a chiral base, for example, (+) or (−) a-methylbenzylamine, or via high performance liquid chromatography using a chiral column.

Platform and scaffold use: In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., “Solid-Supported Combinatorial and Parallel Synthesis of Small-Molecular-Weight Compound Libraries”, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60, 1997). Thus, one embodiment relates to methods of using the compounds described herein for generating derivatives or chemical libraries. The methods can be carried out by performing these, and optionally additional, steps: (1) providing a body comprising a plurality of wells; (2) providing one or more compounds identified by methods described herein in each well (e.g., any of the compounds of Formulas I or II); (3) providing an additional one or more chemicals in each well, where the compound, upon exposure to the chemical(s) may produce one or more products; and (4) isolating the resulting one or more products from each well. We may refer to the original compound as the “first” compound and to the chemical as the “second” compound. The order in which the first and second compounds are added to the wells can vary, and the methods can be carried out in vitro or in cell culture. Lead derivatives can be further tested in animal models.

In alternate embodiments, the methods of using the compounds described herein for generating derivatives or chemical libraries can be carried out using a solid support. These methods can be carried out by, for example: (1) providing one or more of the compounds described herein attached to a solid support; (2) treating the one or more compounds identified by methods described herein attached to a solid support with one or more additional compounds or chemicals; and (3) isolating the resulting one or more products from the solid support. In these methods, “tags” or identifier or labeling moieties may be attached to and/or detached from the compounds described herein or their derivatives, to facilitate tracking, identification or isolation of the desired products or their intermediates. Such moieties are known in the art and exemplary tags are noted above. The chemicals (or “second” compound(s)) used in the aforementioned methods may include, for example, solvents, reagents, catalysts, protecting group and deprotecting group reagents, and the like. Examples of such chemicals are those that appear in the various synthetic and protecting group chemistry texts and treatises which are known in the art and may be referenced herein.

Databases: In one aspect, the invention includes cell-based and in vitro assays (e.g., high throughput screens) that can be used with essentially any compound collection. Following an assay, the result can be recorded in a database, and such databases are also within the scope of the present invention. For example, the invention features a computer-readable database that includes a plurality of records. Each record includes (a) a first field that includes information reflecting the identity of a compound (e.g., a compound within one of the types of libraries described herein) and (b) a second field that includes information concerning the impact of the compound on PARP-1 activity. Additional fields may include the results of toxicity tests, dose-response tests, and the like. The information contained with the fields can be obtained in any order (e.g., the information reflecting PARP-1 activity can be obtained first). However, to help ensure the integrity of the database, the information should be obtained independently (or “blindly”). The database can also include a field comparing the compound to a clinical outcome (e.g., an improvement in a sign or symptom associated with HD, ALS, cancer, or any of the other disorders described herein). The number of records can be, but is not necessarily, great. For example, a useful database can include at least 10, 25, 50, 100, 250, 500, 1000, 1500, 1800, 2000, or 2500 records.

The invention is further illustrated by the following examples, which should not be construed as further limiting.

### EXAMPLES

### Example 1

Identification of compounds that inhibit PARP-1. Mitochondrial dysfunction and ATP deficiency are distinct features of Huntington's disease (HD). Oxidative stress, implicated in HD pathology as an external risk factor, activates a broad range of energy-dependent cellular pathways. The responses of energy-deficient mutant HD cells to oxidative stress may be neither sufficient nor adequate, leading to further decrease of ATP beyond the threshold of normal cell functions and viability. Here we report a loss of ATP at a robust rate in HD cells during oxidative stress, and the prevention of such unsustainable ATP depletion by inhibiting PARP-1 with small molecule inhibitors.

The products of oxidative stress, including various oxidants and free radicals, such as hydrogen peroxide, induce DNA brakes, triggering PARP-1 activation. PARP-1 is one of the most abundant proteins in the nucleus, which can be activated up to 100-fold by damaged DNA. In the absence of functional PARP-1, DNA base excision repair is delayed after exposure of cells to ionizing radiation or alkylating agents. Activated PARP-1 cleaves NAD⁺ into nicotinamide and ADP-ribose, and uses the latter for extensive poly(ADP-ribosyl)ation of histones and other cellular proteins as a part of the DNA repair pathway. Ultimately PARP-1 activation leads to reduction of energy sources, including NAD⁺ and ATP, in the cell. Over-activated PARP-1 may cause necrosis by depleting cell energy sources beyond the survival threshold. In HD cells, where ATP levels are reduced due to mitochondrial impairment, high PARP-1 activity may seriously compromise cellular functions and viability.

To investigate the impact of oxidative stress on HD cells we measured ATP levels in lymphoblasts derived from HD patients and normal controls. ATP levels were measured using a commercially available-luciferase substrate. The luciferase enzyme uses ATP as a cofactor to produce one photon of light per substrate molecule (e.g., luciferin). The detectable output is luminescence. There is a linear relationship between the amount of signal output and the amount of ATP present in the sample. Thus, the amount of ATP present in a sample can be directly correlated with the measured output.

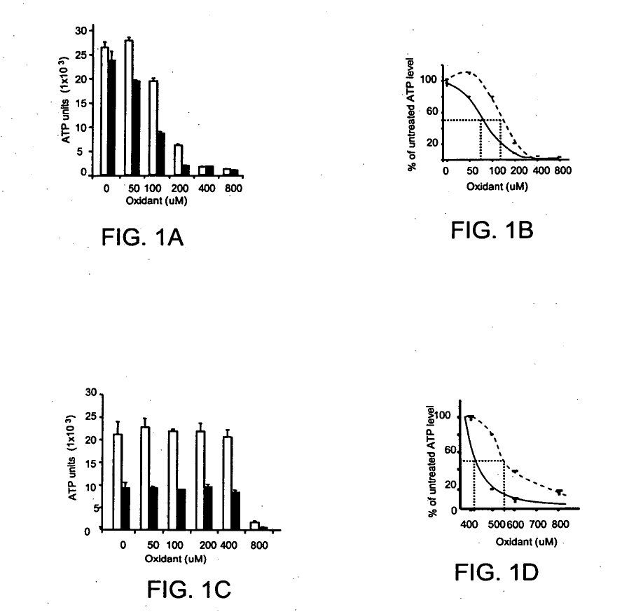

We found that, overall, HD cells had lower then normal basal ATP levels, although the differences between mutant and wildtype levels varied (FIG. 1A). When we treated the cells with the oxidant, hydrogen peroxide, we detected a robust decrease in ATP levels in HD lymphoblasts in response to even moderate oxidative conditions. Under such conditions, ATP levels were sustained in wildtype cells. A 50% decrease in basal ATP levels in HD and wildtype cells was observed at oxidant concentrations of 60 μM and 120 μM, respectively (FIG. 1B). The same phenomenon was observed in experiments with striatal cells, derived from Huntingtin double knock-in mutant and wildtype transgenic mice. Mutant striatal cells demonstrated a greater difference in basal ATP levels than wildtype counterparts (FIG. 1C). Both mutant and wildtype striatum cells showed higher resistance to oxidative stress then lymphoblasts. However, mutant cells showed greater sensitivity than wildtype cells at effective oxidant doses. In cells treated with 500 μM oxidant, we observed a dramatic 80% loss of ATP in mutant cells from basal ATP levels, and only 20% loss of ATP in wildtype cells FIG. 1D. The 50% reductions from basal ATP levels were observed at oxidant concentrations of 410 μM and 550 μM for mutant and wildtype striatal cells respectively.

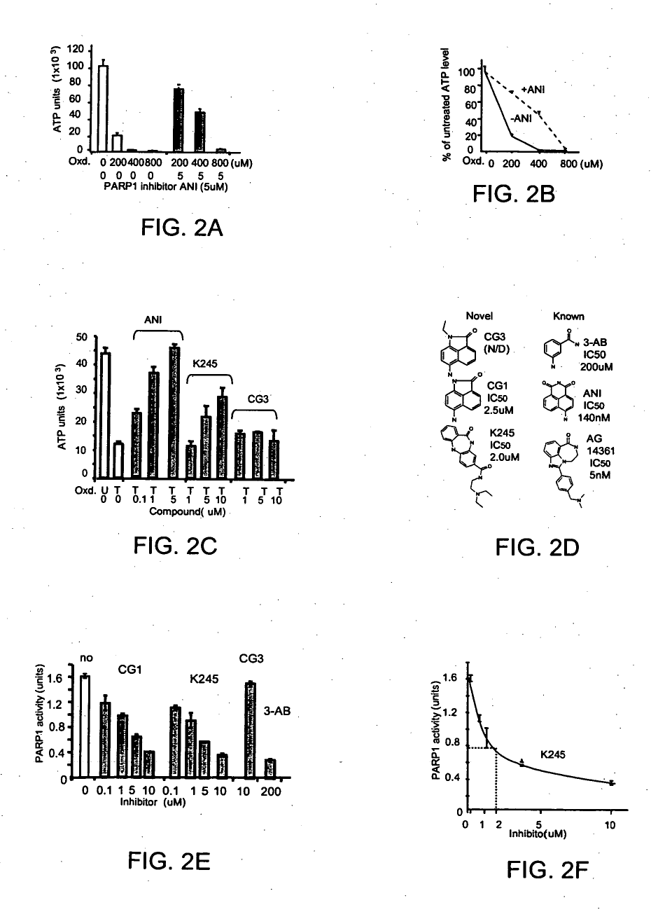

To investigate the basis for the hypersensitivity of mutant cells to oxidative stress, we employed a chemical biological approach and screened for small molecules capable of preventing an unsustainable loss of ATP in HD mutant cells. We developed an assay to measure oxidative stress-dependent ATP loss and subsequent cell-death. We found that a PC 12 cell-line expressing fragments of mutant Htt protein (the fragments consisted of the first 17 amino acids of huntingtin protein followed by 104Q) was hypersensitive to hydrogen peroxide, which even at low doses caused severe ATP depletion, ultimately leading to cell death (FIG. 2A). Using this cell line, we screened a compound library (the MIND compound library (Massachusetts General Hospital, Charlestown, Mass.), which included a commercially available library of natural biologically active compounds from TimTec (Newark, Del.)) and discovered two small molecules (CG1 and K245) that rescued oxidant-mediated ATP loss and cell death (see FIG. 2C, for example). CG1 and K245 have structural properties similar to known PARP1 inhibitors, such as 3-AB, ANI, and AG, suggesting that PARP-1 enzyme was a molecular target for the small molecules (FIG. 2D). Docking of CGI and K245 to the PARP1 active site using the structure prediction program ICM (Abagyan et al., J. Comp. Chem. 15:488-506, 1994) revealed docking score values in the same range as those of known PARP1 inhibitors. In addition, amino groups of each of CGI and K245-14 were predicted to form hydrogen bonds with Ser243 and Gly202 of PARP1. Such interactions have been suggested to be key interactions to facilitate enzyme inhibition (Cepeda et al., Recent Patents on Anti-Cancer Drug Discovery 1:39-53, 2006).

The prediction that CGI and K245 interact with PARP1 to rescue oxidant-mediated ATP loss and cell death was consistent with previous reports of PARP-1 activation in cells exposed to hydrogen peroxide treatment. (See, e.g., Jagtap and Szabo, Nat Rev Drug Discov. 4:421-440, 2005; Ha and Snyder, Proc Natl Acad Sci USA. 96: 13978-13982, 1999; and Ying et al., Proc Natl Acad Sci USA 98:12227-12232, 2001). To test the effect of the compounds on PARP1 activity, an in vitro PARP-1 assay was used. The known PARP-1 inhibitor 3-AB was used as a positive control. The identified compounds were found to have a dose-dependent inhibitory effect (FIG. 2E). The IC₅₀S of CG1 and K245 for PARP-1 inhibition in vitro were determined to be 2.5 μM and 2.0 μM, respectively (FIG. 2F and Table 3, below). CG3 was not observed to function as a PARP-1 inhibitor in in vitro or in vivo assays. CG3 has been categorized as a general transcriptional activator, but despite apparent structural similarity to the PARP-1 inhibitor CG1, CG3 did not show any PARP-1 inhibition activity and did not protect cells from oxidative stress. Thus, CG3 was used as a negative control in this study.

To confirm that inhibition of PARP-1 activity could protect cells from ATP depletion, we tested two known enzyme inhibitors in PC12 cells challenged with oxidative stress. The PC12 cell line expressed fragments of mutant Htt protein as described above. Both of the known enzyme inhibitors prevented loss of ATP in a concentration-dependent manner, although ANI, the most potent inhibitor (IC₅₀=140 nM), protected high levels of cellular ATP (see FIGS. 2A, 2B, and 2C). Maintenance of ATP levels rescued by the PARP-1 inhibitors was dependent on the severity of cellular oxidation (FIG. 2A).

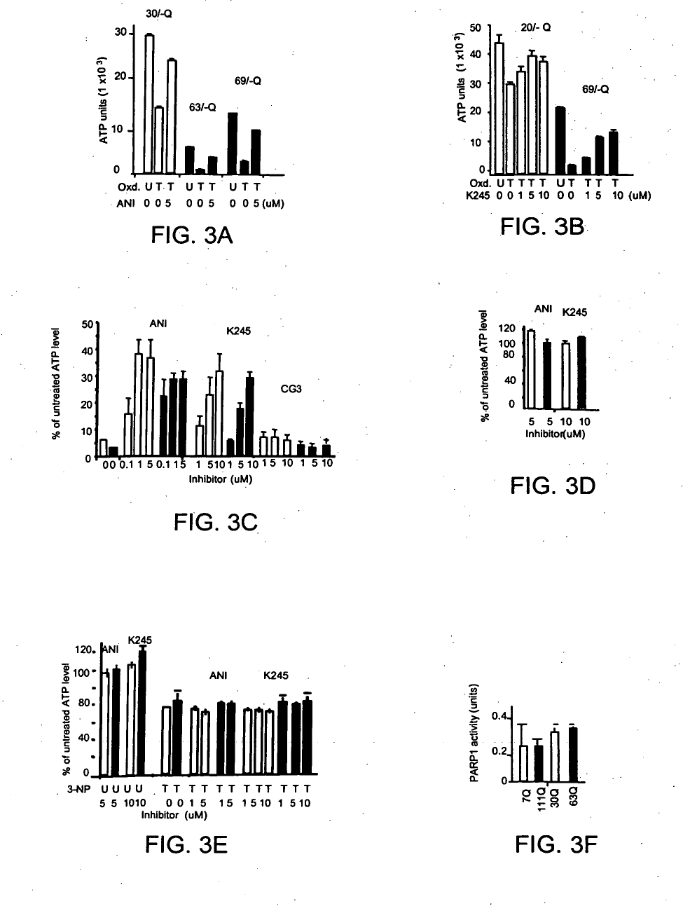

We also determined that the most effective PARP-1 inhibitors, ANI and K245, rescued loss of ATP in Huntington's disease (HD) and wild type lymphoblasts, and in mutant and wild type striatal cells (FIGS. 3A, 3B, and 3C). The inhibitors protected loss of ATP in a concentration-dependent manner and also retained absolute residual ATP levels. The maintenance of ATP levels was dependent on compound potencies, severity of the stress modeled by oxidant concentrations, and the basal ATP levels in unstressed cells. We measured and detected no effects of PARP-1 inhibitors on basal PARP-1 activities in normal and HD lymphoblasts (FIG. 3D), or in wild type and mutant striatal cells (FIG. 3F). Notably, the identified inhibitors were ineffective to prevent ATP loss mediated by mitochondrial blocker 3-nitropropionic acid (3-NP), demonstrating specificity of the inhibitors for molecular target PARP-1, un-induced by 3-NP.

We also measured and detected no effects of PARP-1 inhibitors on PARP-1 basal activities in normal and HD lymphoblasts (FIG. 3D) or in wild type and mutant striatal cells (FIG. 3F). We also did not detect significant differences in PARP-1 protein levels in lymphoid cell lines from HD patients and normal individuals, or in mutant and wild type striatal cell lines derived from knock-in mice. However, basal PARP-1 expression levels were low in both mutant and wild type striatal cell lines, which might explain their resistance to high concentrations of oxidant.

The results described above show that PARP-1 inhibitors effectively protected ATP-deficient HD cells from energy depletion mediated by oxidative stress.

### Example 2

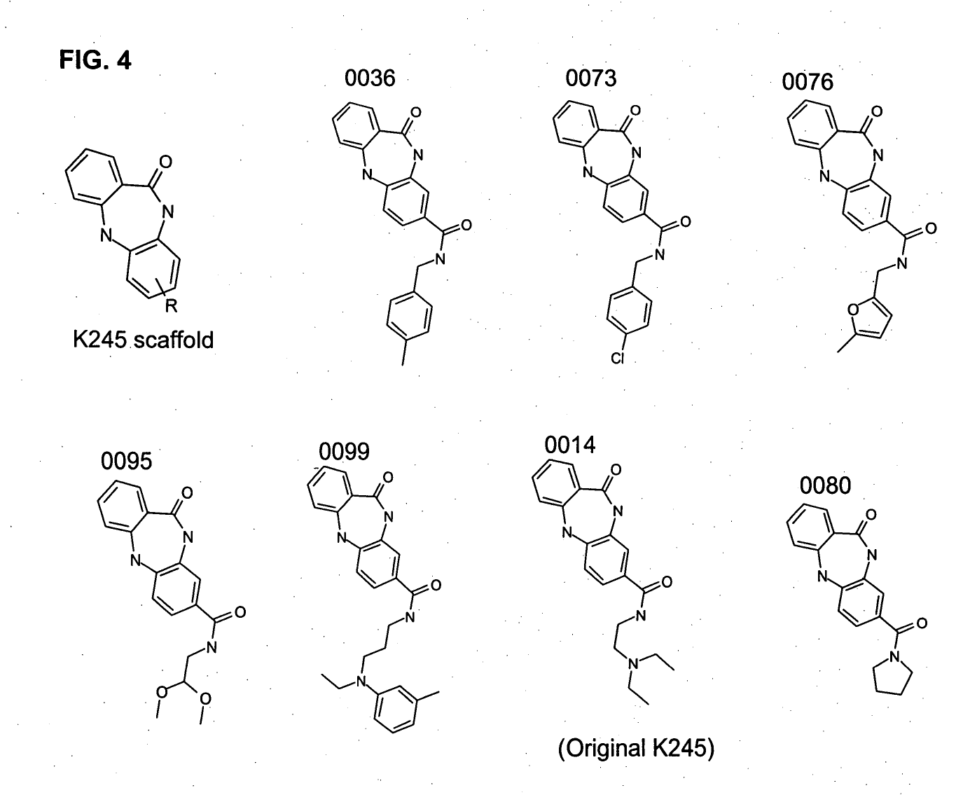

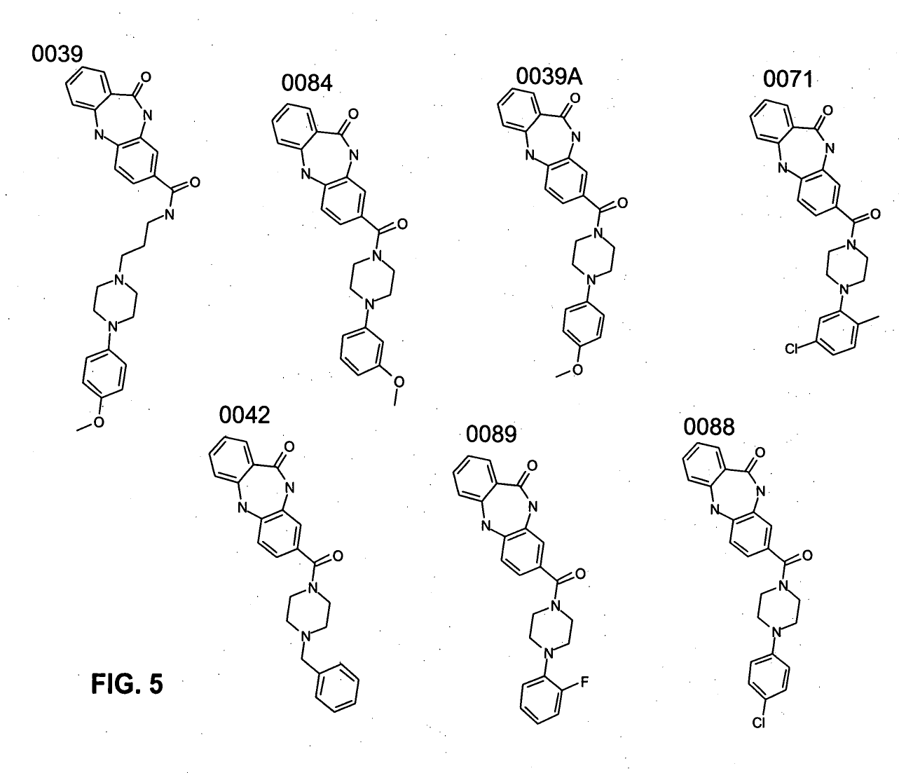

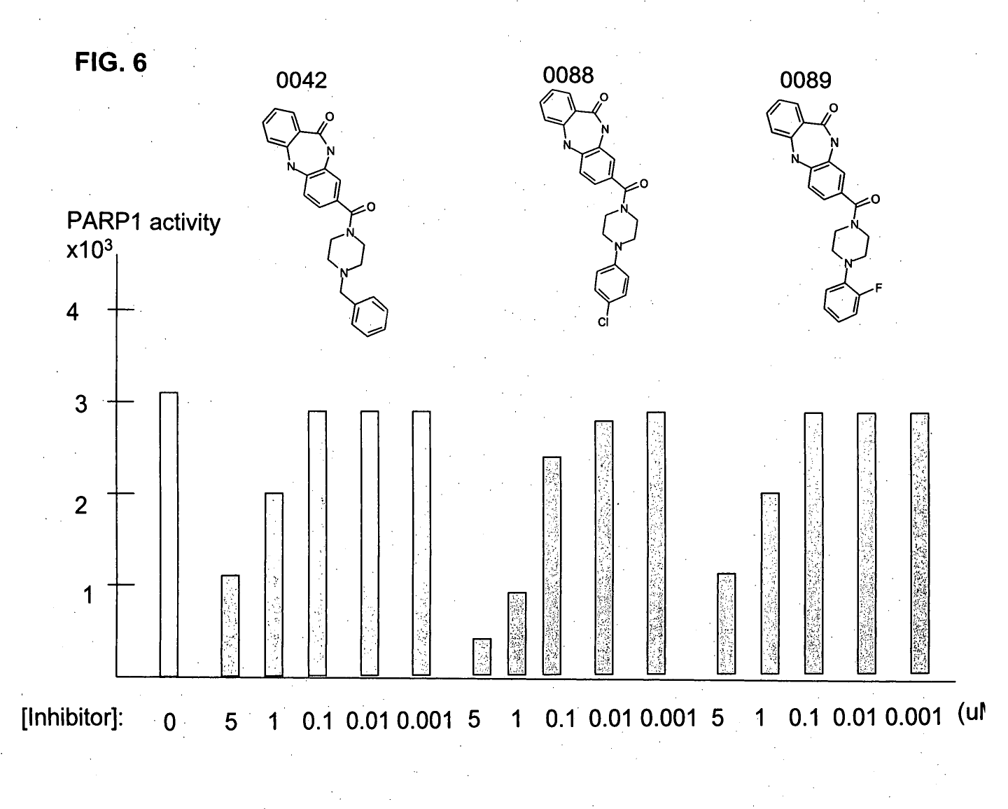

Identification of structural analogs. Structure activity relationship (SAR) studies were conducted with the novel K245 PARP-1 inhibitor scaffold structure, and several analogs were identified. Structures of the analog compounds are shown in FIGS. 4 and 5. Docking studies of these molecules provided positions and docking score values similar to those of K245-14 indicating that the molecules are likely to bind to PARP1. In in vitro enzyme inhibition assays, all the molecules demonstrated PARP1 inhibition, with the best inhibitor demonstrating a 10-fold increase in potency over the original K245-14 compound (Table 3). PARP-1 activity in the presence of decreasing concentrations of three different inhibitors is shown in the graph in FIG. 6.

TABLE 3
PARP1 inhibitor binding characteristics

| | | Predicted | Predicted | |
|---|---|---|---|---|
| PARP1 | ICM | binding | binding | |
| inhibitor | score | energy | affinity | IC50 |
| ID | [units] | [kcal] | [μMol] | (μM) |
| 3-AB | −37.62 | −7.55 | 4.0 | 200 |
| 4-ANI | −45.14 | −7.93 | 2.2 | 0.14 |
| CG1 | −42.94 | −7.82 | 2.6 | 2.5 |
| K245-14 | −70.77 | −9.21 | 0.26 | 2.0 |
| K245-42 | −68.89 | −9.12 | 0.30 | 2.2 |
| K245-39A | −57.55 | −8.55 | 0.78 | 5.5 |
| K245-80 | −55.76 | −8.46 | 0.90 | 3.5 |
| K245-88 | −63.33 | −8.84 | 0.48 | 0.15 |
| K245-89 | −59.56 | −8.65 | 0.66 | 0.55 |
| K245-95 | −63.07 | −8.83 | 0.49 | 2.0 |
| K245-99 | −90.19 | −10.19 | 0.053 | 4.0 |

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A pharmaceutical composition comprising a compound of formula (I): 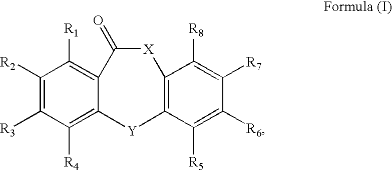
wherein
each of X and Y, independently, is O or NR₉;
each of R₁, R₂, R₃, R₄, R₅, R₆, R₇, and R₈, independently, is R₁₀, halo, NR₁₁R₁₂, OR₁₀, C(O)R₁₀, C(O)OR₁₀, C(O)NR₁₁R₁₂, CN, or NO₂;
R₉, independently, is H, alkyl, cycloalkyl, heterocycloalkyl, aryl, heteroaryl, or C(O)R₁₀;
R₁₀, independently, is H, alkyl, cycloalkyl, heterocycloalkyl, aryl, or heteroaryl;
each of R₁₁ and R₁₂ is, independently, H, alkyl, cycloalkyl, heterocycloalkyl, aryl, heteroaryl;
or R₁₁ and R₁₂ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl.

2. The composition of claim 1, wherein X is NR₉.

3. The composition of claim 1, wherein Y is NR₉.

4. The composition of claim 3, wherein X is NR₉.

5. The composition of claim 4, wherein each of R₁, R₂, R₃, R₄, R₅, R₆, and R₈ is H.

6. The composition of claim 5, wherein R₇ is C(O)NR₁₁R₁₂.

7. The composition of claim 6, wherein each of X and Y is NH.

8. The composition of claim 7, wherein R₁₁ is H, and R₁₂ is C₁₋₆ alkyl optionally substituted with aryl, heteroaryl, alkoxy, amino, cycloalkyl, heterocycloalkyl, carbonyl, carboxy, or alkoxycarbonyl.

9. The composition of claim 7, wherein R₁₁ and R₁₂ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl.

10. The composition of claim 9, wherein R₁₁ and R₁₂ together with the nitrogen atom to which they are attached are piperazin-1-yl or pyrrolidin-1-yl, each of which is optionally substituted with alkyl, cycloalkyl, heterocycloalkyl, aryl, heteroaryl, alkoxy, amino, or carbonyl, or optionally fused with cycloalkyl, heterocycloalkyl, aryl, or heteroaryl.

11. The composition of claim 6, wherein R₁₁ is H, and R₁₂ is C₁₋₆ alkyl optionally substituted with aryl, heteroaryl, alkoxy, amino, cycloalkyl, heterocycloalkyl, carbonyl, carboxy, or alkoxycarbonyl.

12. The composition of claim 6, wherein R₁₁ and R₁₂ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl.

13. The composition of claim 12, wherein R₁₁ and R₁₂ together with the nitrogen atom to which they are attached are piperazin-1-yl or pyrrolidin-1-yl, each of which is optionally substituted with alkyl, cycloalkyl, heterocycloalkyl, aryl, heteroaryl, alkoxy, amino, or carbonyl, or optionally fused with cycloalkyl, heterocycloalkyl, aryl, or heteroaryl.

14. The composition of claim 1, wherein R₇ is C(O)NR₁₁R₁₂.

15. The composition of claim 14, wherein R₁₁ is H, and R₁₂ is C₁₋₆ alkyl optionally substituted with aryl, heteroaryl, alkoxy, amino, cycloalkyl, heterocycloalkyl, carbonyl, carboxy, or alkoxycarbonyl.

16. The composition of claim 14, wherein R₁₁ and R₁₂ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl.

17. The composition of claim 1, wherein one of R₁, R₂, R₃, R₄, R₅, R₆, R₇, and R₈ is C(O)NR₁₀R₁₁, and the others are H.

18. The composition of claim 1, wherein the compound is

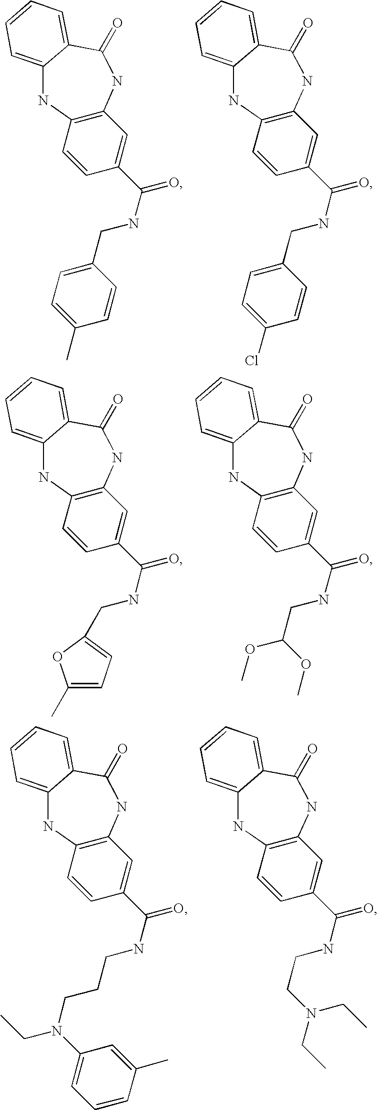

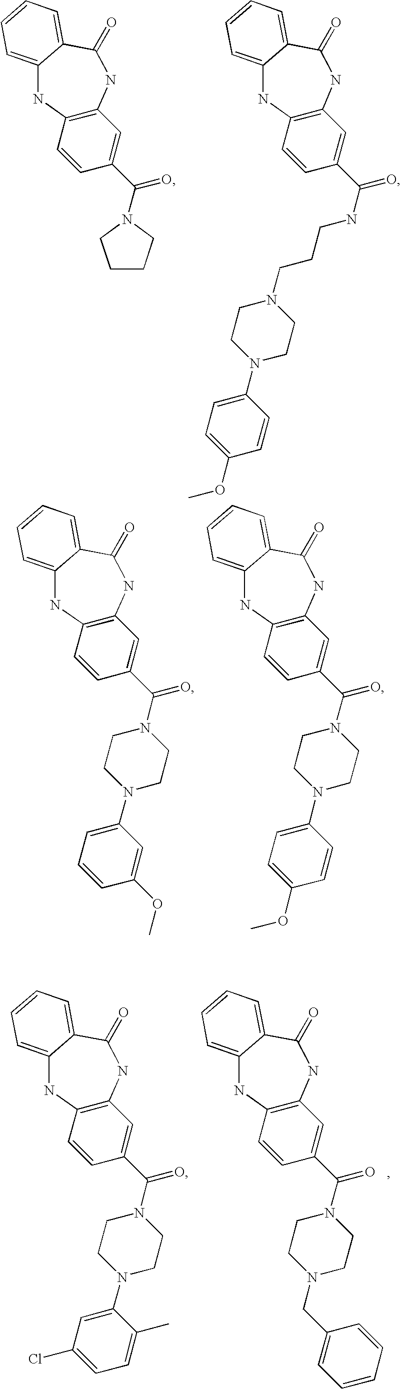

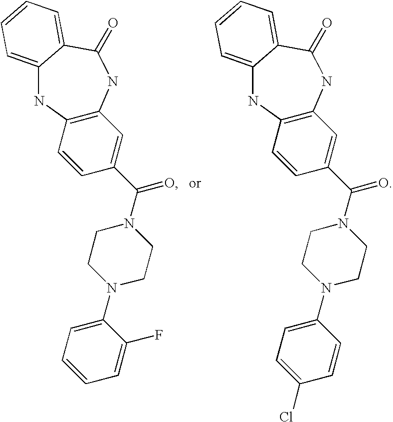

19. A pharmaceutical composition comprising a compound of formula (II): 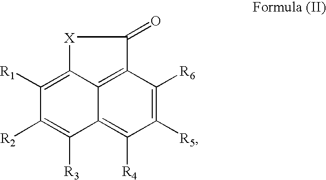
wherein
X is O or NR₇;
each of R₁, R₂, R₃, R₄, R₅, and R₆, independently, is R₈, halo, NR₉R₁₀, OR₈, C(O)R₈, C(O)OR₈, C(O)NR₉R₁₀, CN, or NO₂;
R₇ is H, alkyl, cycloalkyl, heterocycloalkyl, aryl, heteroaryl, or C(O)R₁₀;
R₈, independently, is H, alkyl, cycloalkyl, heterocycloalkyl, aryl, or heteroaryl;
each of R₉ and R₁₀ is, independently, H, alkyl, cycloalkyl, heterocycloalkyl, aryl, heteroaryl;
or R₉ and R₁₀ together with the nitrogen atom to which they are attached form a 3-8 membered ring containing 1-3 heteroatoms, the ring being optionally substituted with alkyl, cycloalkyl, heterocycloalkyl, aryl, heteroaryl, alkoxy, amino, or carbonyl, or the ring being optionally fused with cycloalkyl, heterocycloalkyl, aryl, or heteroaryl.

20. The composition of claim 19, wherein X is NR₇.

21. The composition of claim 20, wherein each of R₁, R₂, R₄, R₅, and R₆ is H.

22. The composition of claim 21, wherein R₃ is NR₉R₁₀.

23. The composition of claim 22, wherein each of R₉ and R₁₀ is H.

24. The composition of claim 23, wherein R₇ is H or alkyl.

25. The composition of claim 20, wherein R₇ is H or alkyl.

26. The composition of claim 19, wherein one of R₁, R₂, R₃, R₄, R₅, and R₆ is NR₉R₁₀, and the others are H.

27. The composition of claim 19, wherein the compound is

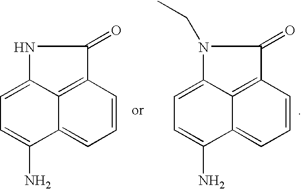

28. A method of treating a subject who has been diagnosed as having, or is at risk of developing, a disorder characterized by poly(ADP-ribose)polymerase 1 (PARP-1) activity, the method comprising identifying the subject and administering to the subject a therapeutically effective amount of the pharmaceutical composition of claim 1.

29. The method of claim 28, wherein the subject has been diagnosed as having, or is at risk for developing, Huntington's Disease.

30. The method of claim 28, wherein the subject has been diagnosed as having or is at risk for developing, Amyotrophic Lateral Sclerosis (ALS), Alzheimer's disease, Parkinson's disease, hereditary hemochromatosis, atherosclerosis, Ewing's sarcoma, high-grade lymphoma, circulatory shock, or colitis.

31. The method of claim 28, wherein the subject has had or is at risk for having a stroke or myocardial infarction.

32. The composition of claim 1, wherein the compound has an IC₅₀ of 0.10 to 6.0 μM.

33. The composition of claim 19, wherein the compound has an IC₅₀ of 0.10 to 6.0 μM.
